(19) United States
(12) Patent Application Publication
(10) Pub. No.: US 2011/0268810 A1
(43) Pub. Date: Nov. 3, 2011

(54) POLYMERIC MATERIALS LOADED WITH MUTAGENIC AND RECOMBINAGENIC NUCLEIC ACIDS

(75) Inventors: William Mark Saltzman, New Haven, CT (US); Peter M. Glazer, Guilford, CT (US); Joanna Chin, Douglaston, NY (US); Nicole McNeer, Westport, CT (US)

(73) Assignee: Yale University

(21) Appl. No.: 12/938,177

(22) Filed: Nov. 2, 2010

Related U.S. Application Data
(60) Provisional application No. 61/257,135, filed on Nov. 2, 2009.

(51) Int. Cl. A61K 9/14; A61K 48/00; C12N 15/87; B82Y 5/00

(52) U.S. Cl. 424/499; 435/455; 514/44 R; 977/773; 977/916

(57) ABSTRACT

Polymeric microparticles are used to deliver recombinagenic or mutagenic nucleic acid molecules such as donor nucleic acid alone, or in combination with triplex-forming molecules, to induce a site-specific mutation in the target DNA. Target cells endocytose the particles, releasing the nucleic acid molecules inside of the cell, where they induce mutagenesis or recombination at a target site. The examples demonstrate that triplex forming oligonucleotides, preferably PNAs, preferably in combination with a donor nucleotide molecule, can be encapsulated into polymeric microparticles, which are delivered into cells. Results demonstrate significantly greatly levels of uptake and expression, and less cytotoxicity, as compared to direct transfer of the nucleic acid molecules into the cell by nucleofection.

### CROSS-REFERENCE TO RELATED APPLICATIONS

This application claims priority under 35 U.S.C. 119 to U.S. Ser. No. 61/257,135 filed Nov. 2, 2009.

### STATEMENT REGARDING FEDERALLY SPONSORED RESEARCH

The U.S. government has certain right in this invention by virtue of grants from the National Institutes of Health EB000487 to William Mark Saltzman and HL 082655 to Peter M. Glazer. This work was also supported by NIGMS Medical Scientist Training Program T32GM07205 (N.A.M. and J.Y.C).

### FIELD OF THE INVENTION

The present invention relates to polymer microparticles for delivery of donor DNA nucleic acid molecules that recombine with genomic DNA for site specific modification, alone or in combination with triplex forming oligonucleotides, with higher efficiency and lower cytotoxicity than other methods.

### BACKGROUND OF THE INVENTION

PNAs contain nucleobases with a peptide-like backbone, making them resistant to both proteases and nucleases, and giving PNA/DNA complexes increased stability compared to DNA/DNA complexes due to the lack of negatively charged phosphodiester bonds. PNAs can form a triplex structure with DNA by strand invasion, triggering DNA repair and thereby stimulating recombination of short donor DNA fragments near the PNA's binding site. bis-PNA-194 (IVS2-194), which targets a polypurine site in the second intronic sequence of the human β-globin gene, can stimulate site-specific gene modification when co-introduced with a short, single-stranded donor DNA encoding the desired modification. PNAs do not readily cross the cell membrane, so special delivery methods are required. The Amaxa nucleofection/electroporation system has been established as a superior method of DNA transfection for hematopoietic stem cells. In earlier studies, the oligonucleotides were introduced into human progenitor cells using the Amaxa (also called Lonza) commercially available nucleofector kit, which is somewhat toxic to cells, and cannot be used in vivo.

Alternatives to nucleofection for PNA have been tested, but all have serious drawbacks. Cationic liposome delivery protocols for PNA usually employ complementary carrier DNA to provide a negative charge, but this application required the use of non-complementary and non-conjugated PNA/donor DNA combinations due to the distance between the PNA and DNA binding sites. Other methods of PNA delivery include microinjection, conjugation to cell-penetrating peptides, and conjugation to lipophilic moieties. Some recently developed methods of PNA delivery have been successful, but rely on covalent modification of the PNA or complexation with complementary DNA, or the use of non-biodegradable materials. Importantly, the majority of studies on PNA delivery have been conducted in cell line reporter systems, which are relatively easy to transfect in comparison to the CD34⁺ hematopoietic progenitors that are targets for clinical applications.

In addition to challenges in PNA delivery, delivery of single-stranded nucleic acids for therapeutic use remains an active area of research. Even for conventional nucleic acids, gene delivery into human hematopoietic progenitors presents many challenges, and many studies have relied on the use of electroporation, nucleofection, or microinjection. Other researchers have explored non-viral methods for the genome modification of human hematopoietic and immune cells using strategies ranging from small fragment homologous replacement to zinc finger nucleases.

It is therefore an object of the present invention to provide a gentle and versatile delivery system which can preferentially deliver nucleic acid molecules to selected cells or tissue, with high efficiency and minimal toxicity.

### SUMMARY OF THE INVENTION

Polymeric microparticles are used to deliver donor nucleic acid molecules, alone or in combination with triplex forming oligonucelotides, to induce a site-specific mutation in the target DNA. Target cells endocytose the particles, releasing the nucleic acid molecules inside of the cell, where they bind to the target to be mutated. Targeting molecules can also be attached to the surface of the microparticles to increase specificity and uptake efficiency. Specificity is determined through the selection of the targeting molecules. The effect can also be modulated through the density and means of attachment, whether covalent or ionic, direct or via the means of linkers.

The examples demonstrate that a donor nucleotide molecule, preferably in combination with triplex-forming molecules such as PNAs, can be encapsulated into polymeric microparticles, which are delivered into cells. Results demonstrate significantly greatly levels of uptake and expression, and less cytotoxicity, as compared to direct transfer of the nucleic acid molecules into the cell by nucleofection.

### BRIEF DESCRIPTION OF THE DRAWINGS

FIG. 1 is a bar graph showing the DNA and PNA content (pmoles nucleic acid/mg nanparticles) content of PLGA nanoparticles loaded with 1 nmole DNA +13.5 ug spermidine/mg PLGA (“DNA”), 0.5 nmole PNA +0.5 nmole DNA/mg PLGA (“PNA−DNA”), or 1 nmole PNA/mg PLGA (“PNA”). Loading of PNA and DNA per mg of nanoparticles is given +/− standard deviation, n=4 for each batch. The percent of the loaded nucleic acid released after 24 hours for each group is expressed as a percentage below the graph.

FIG. 2A is a bar graph showing the uptake of nanoparticles (cell associated fluorescence in arbitrary fluorescence units) for untreated control cells and cells treated with 0.2 mg/ml or 2.0 mg/ml fluorescent dye coumarin 6 (C6) nanoparticles, after 1 or 3 days. FIGS. 2B, 2C, and 2D show the uptake of nanoparticles (cell associated fluorescence in arbitrary units) for untreated control cells and cells treated with 1×10⁵ or 1×10⁶ nanoparticles/cell, after 1, 3, or 5 days respectively. Nanoparticles are unmodified or with antennapedia peptide (“AP”); % internalized as indicated beneath the graphs. FIG. 2D shows cells repeated 1:5.

FIG. 3A is a histogram showing CD34+ cells (% of Max) with internal fluorescent dye coumarin 6 (C6) nanoparticles as function of fluorescence intensity (FL1-H, log scale). FIG. 3B is a histogram showing CD34+ cells (# of cells) with internal fluorescent dye coumarin 6 (C6) nanoparticles as function of fluorescence intensity (FL1-H, log scale). Cells were treated with 1×10⁵ or 1×10⁶ nanoparticles/cell; nanoparticles are unmodified or with antennapedia peptide (“AP”).

FIGS. 4A and 4B are bar graphs showing cell survival (cells per 100 original cells) one day (Day 1) and three days (Day 3) respectively, after treatment with PLGA nanoparticle with or without nucleic acid loading, or nucleofection with nucleic acid, or mock nucleofected, or untreated. Counts are normalized to original cell platings. Error bars for live and dead cells give standard deviation where available. **p=0.01, ***p=5×10⁻¹².

FIGS. 4C, 4D, and 4E are bar graphs showing the cells survival (total live cells) for untreated control cells and cells treated with 1×10⁵ or 1×10⁶ nanoparticles/cell, after 1, 3, or 5 days respectively. Nanoparticles are unmodified or with antennapedia peptide (“AP”); % dead cells as indicated beneath the graphs.

FIG. 5A is a schematic showing bis-PNA stand-displacement and triplex formation at a target site on a DNA duplex. FIG. 5B is a schematic of the PNA−DNA model system used to investigate nucleic acid loaded nanoparticle-mediated stimulation of genomic recombination to modify the IVS2-1 splice site within the beta-globin gene.

FIG. 6 is a bar graph showing dose-dependent modification of the β-globin gene (modification (day 7) in arbitrary units) in cells treated with high, medium, or low doses of nanoparticles containing both PNA and donor DNA together (PNA−DNA); or two species of nanoparicles: PNA and donor DNA separately (PNA+DNA); or nanoparticles with DNA alone. Error bars where indicated give +/− standard deviation (n=3). Expression of the mutant is given in arbitrary units, with normalization to expression of the β-globin wildtype allele. Dosages are expressed as nmoles of nucleic acid/mL of media based on a particle loading of approximately 1 nmole nucleic acid/mg particles. For example, for “low” dose: 0.5 nmoles of DNA per mL media, or 0.5 nmoles DNA +0.5 nmoles PNA per mL media, based on particle loading, which corresponds to 0.5 mg/mL DNA particles, 0.5 mg/mL DNA particles +0.5 mg/mL PNA particles, or 1 mg/mL PNA−DNA particles. “Medium”: 1 nmoles DNA per mL media, or 1 nmole DNA +1 nmole PNA per mL media. “High”: 2 nmoles DNA per mL media, or 2 nmole DNA +2 nmole PNA per mL media.

FIG. 7 is a graph showing the mutant allele frequency (%) of PNA+DNA nanoparticle-treated CD34+ genomic DNA (-▪-), PNA+DNA nucelofected CD34+genomic DNA (-▴-), and (--) wildtype CD34+gDNA spiked with ssDNA donor oligo as a function of mutant primer qPCR (Normalized to wildtype AS-PCR, arbitrary units) plotted using a standard curve (mutant plasmid+wildtype CD34++gDNA) generated by quantitative AS-PCR with known amounts of mutant plasmid copies.

FIG. 8 is a schematic depiction of a limiting/low dilution assay to independently determine modification frequency.

### DETAILED DESCRIPTION OF PREFERRED EMBODIMENTS

### I. Polymeric Microparticles

The term “microparticle” includes “nanoparticles” unless otherwise stated. As used herein, microparticles generally refers to both microparticles in the range of between 0.5 and 1000 microns and nanoparticles in the range of between 50 nm to less than 0.5, preferably having a diameter that is between 1 and 20 microns or having a diameter that is between 50 and 500 nanometers, respectively. Microparticles and nanoparticles are also referred to more specifically.

The external surface of the microparticles may be modified by conjugating to the surface of the microparticle a coupling agent or ligand. As described below, in the preferred embodiment, the coupling agent is present in high density on the surface of the microparticle.

The microparticle may be further modified by attachment of one or more different molecules to the ligands or coupling agents, such as targeting molecules, attachment molecules, and/or therapeutic, nutritional, diagnostic or prophylactic agents.

A targeting molecule is a substance which will direct the microparticle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

Surface modified matrices as referred to herein present target that facilitate attachment of cells, molecules or target specific macromolecules or particles.

By varying the polymer composition of the particle and morphology, one can effectively tune in a variety of controlled release characteristics allowing for moderate constant doses over prolonged periods of time. There have been a variety of materials used to engineer solid nanoparticles with and without surface functionality (as reviewed by Brigger et.al Adv Drug Deliv Rev 54, 631-651 (2002)). Perhaps the most widely used are the aliphatic polyesters, specifically the hydrophobic poly (lactic acid) (PLA), more hydrophilic poly (glycolic acid) PGA and their copolymers, poly (lactide-co-glycolide) (PLGA). The degradation rate of these polymers, and often the corresponding drug release rate, can vary from days (PGA) to months (PLA) and is easily manipulated by varying the ratio of PLA to PGA. Second, the physiologic compatibility of PLGA and its hompolymers PGA and PLA have been established for safe use in humans; these materials have a history of over 30 years in various human clinical applications including drug delivery systems. Finally, PLGA nanoparticles can be formulated in a variety of ways that improve drug pharmacokinetics and biodistribution to target tissue by either passive or active targeting.

A. Polymers

Non-biodegradable or biodegradable polymers may be used to form the microparticles. In the preferred embodiment, the microparticles are formed of a biodegradable polymer. Non-biodegradable polymers may be used for oral administration. In general, synthetic polymers are preferred, although natural polymers may be used and have equivalent or even better properties, especially some of the natural biopolymers which degrade by hydrolysis, such as some of the polyhydroxyalkanoates. Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the microparticles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof.

In a preferred embodiment, PLGA is used as the biodegradable polymer.

The microparticles are designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. Specifically the hydrophobic poly (lactic acid) (PLA), more hydrophilic poly (glycolic acid) PGA and their copolymers, poly (lactide-co-glycolide) (PLGA) have various release rates. The degradation rate of these polymers, and often the corresponding drug release rate, can vary from days (PGA) to months (PLA) and is easily manipulated by varying the ratio of PLA to PGA.

B. Formation of Microparticles

In addition to the preferred method described in the examples for making microparticles, there may be applications where microparticles can be fabricated from different polymers and/or using different methods.

Solvent Evaporation. In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microparticles. The resulting microparticles are washed with water and dried overnight in a lyophilizer. Microparticles with different sizes (0.5-1000 microns) and morphologies can be obtained by this method. This method is useful for relatively stable polymers like polyesters and polystyrene.

In the preferred embodiment, the molecules to be delivered are encapsulated into the polymer using double emulsion solvent evaporation techniques, such as that described by Luo et al., Controlled DNA delivery system, Phar. Res., 16: 1300-1308 (1999). The polymer is dissolved in an organic solvent such as methylene chloride or ethyl acetate (GRAS solvents are preferred), DNA is added, the solution vortexed and chilled, and the solvent removed by evaporation, preferably while frozen.

Solvent Extraction or Removal. In this method, the nucleic acid molecules are dispersed in a solution of the selected polymer in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. Microparticles that range between 1-300 microns can be obtained by this procedure. The external morphology of spheres produced with this technique is highly dependent on the type of polymer used.

Spray-Drying In this method, the polymer is dissolved in organic solvent. A known amount of the nucleic acid molecules are suspended in the polymer solution. The dispersion is then spray-dried. Typical process parameters for a mini-spray drier (Buchi) are as follows: polymer concentration=0.04 g/mL, inlet temperature=−24° C., outlet temperature=13-15 ° C., aspirator setting=15, pump setting=10 mL/minute, spray flow=600 Nl/hr, and nozzle diameter=0.5 mm. Microparticles ranging between 1-10 microns are obtained with a morphology which depends on the type of polymer used.

Hydrogel Microparticles. Microparticles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath for twenty to thirty minutes in order to allow sufficient time for gelation to occur. Microparticle particle size is controlled by using various size extruders or varying either the nitrogen gas or polymer solution flow rates. Chitosan microparticles can be prepared by dissolving the polymer in acidic solution and crosslinking it with tripolyphosphate. Carboxymethyl cellulose (CMC) microparticles can be prepared by dissolving the polymer in acid solution and precipitating the microparticle with lead ions. In the case of negatively charged polymers (e.g., alginate, CMC), positively charged ligands (e.g., polylysine, polyethyleneimine) of different molecular weights can be conically attached.

### II. Triplex Forming Molecules, Donor Molecules, Fusions

There are two principle groups of molecules to be encapsulated or attached to the polymer, either directly or via a coupling molecule: targeting molecules, attachment molecules and triplex forming nucleic acid molecules. These can be coupled to the surface and/or encapsulated using standard techniques.

A. Triplex-Forming Molecules

Disclosed herein are compositions containing molecules, referred to as “triplex-forming molecules”, that bind to duplex DNA in a sequence-specific manner to form a triple-stranded structure. The triplex-forming molecules can be used to induce site-specific homologous recombination in mammalian cells when combined with donor DNA molecules.

The predetermined region that the triplex-forming molecules bind to is referred to herein as the “target sequence”, “target region”, or “target site”. Target sequences can be within the coding DNA sequence of the gene or within introns. Target sequences can also be within DNA sequences which regulate expression of the target gene, including promoter or enhancer sequences. Preferably, the target sequence of the triplex-forming molecule is within or is adjacent to a human gene.

The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids.

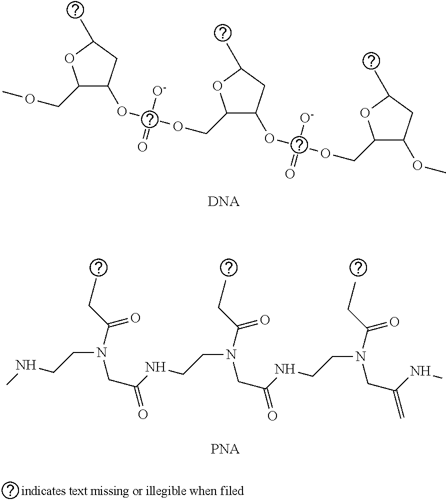

1. Triplex-Forming Oligonucleotides (TFOs)

In one embodiment, the triplex-forming molecules are triplex-forming oligonucleotides. Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined region of a double-stranded DNA molecule so as to form a triple-stranded structure.

Preferably, the target region of the double-stranded molecule contains or is adjacent to a defective or essential portion of a target gene, such as the site of a mutation causing a genetic defect, a site causing oncogene activation, or a site causing the inhibition or inactivation of an oncogene suppressor. More preferably, the gene is a human gene.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful.

The oligonucleotides are preferably generated using known DNA synthesis procedures. In one embodiment, oligonucleotides are generated synthetically. As discussed below, oligonucleotides can also be chemically modified using standard methods that are well known in the art.

The nucleotide sequence of the oligonucleotides is selected based on the sequence of the target sequence, the physical constraints imposed by the need to achieve binding of the oligonucleotide within the major groove of the target region, and the need to have a low dissociation constant (K_{d}) for the oligonucleotide/target sequence. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

Preferably, the oligonucleotide binds to or hybridizes to the target sequence under conditions of high stringency and specificity. Most preferably, the oligonucleotides bind in a sequence-specific manner within the major groove of duplex DNA. Reaction conditions for in vitro triple helix formation of an oligonucleotide probe or primer to a nucleic acid sequence vary from oligonucleotide to oligonucleotide, depending on factors such as oligonucleotide length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, triplex-forming molecules are said to be substantially complementary to a target region when the molecules have a heterocyclic base composition which allows for duplex strand displacement and the formation of a triple-helix with the target region. As such, triplex-forming molecules are substantially complementary to a target region even when there are non-complementary bases present in the molecules. There are a variety of structural motifs available which can be used to determine the nucleotide sequence of the substantially complementary molecules.

2. Peptide Nucleic Acids

Some triplex forming molecules, for example, peptide nucleic acids (PNAs), are a pair of single-stranded molecules, or a pair of molecules connected by a linker, that facilitate strand displacement and triplex formation, referred to as a “clamp,” in which one molecule binds to the target strand by Hoogsteen binding and the other molecule binds to the target strand by Watson-Crick binding in a sequence specific manner. As used herein, the pair of single-stranded triplex-forming molecules may be referred to individually as the Watson-Crick binding portion, and the Hoogsteen binding portion. As described below, some triplex-forming molecules also have a Watson-Crick binding “tail” added to the end of the Watson-Crick binding portion of the clamp. The “tail” includes additional nucelobases that bind to the target strand outside the triple helix formed at the site of duplex strand displacement. In one preferred embodiment, the triplex-forming molecules are two PNA molecules, the Watson-Crick portion includes a tail, and the two PNA molecules are linked by an O-linker.

Peptide nucleic acids are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are comprised of peptide nucleic acid monomers. The heterocyclic bases can be any of the standard bases (uracil, thymine, cytosine, adenine and guanine) or any of the modified heterocyclic bases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules, or two PNA molecules linked together by a linker of sufficient flexibility to form a bis-PNA. In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the DNA−PNA duplex (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker molecule monomers in any combination.

a. Tail Clamp

Although polypurine:polypyrimidine stretches do exist in mammalian genomes, it is desirable to target triplex formation in the absence of this requirement. Some triplex-forming molecules include a “tail” added to the end of the Watson-Crick binding portion. Adding additional nucleobases, known as a “tail” or “tail clamp”, to the Watson-Crick binding portion that bind to target strand outside the triple helix further reduces the requirement for a polypurine:polypyrimidine stretch and increases the number of potential target sites. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix and the PNA/DNA duplex both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. USA., 99(26):16695-700 (2002)). Tail clamps added to PNAs (referred to as tcPNAs) have been described by Kaihatsu, et al., Biochemistry, 42(47):13996-4003 (2003); Bentin, et al., Biochemistry, 42(47):13987-95 (2003), and are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site.

b. Targeting and Sequence Considerations for PNAs

The tail-clamp bis-PNAs are designed to target a specific sequence of the target duplex nucleotide. The nucleotide sequences of the triplex-forming molecules are selected based on the sequence of the target sequence, the physical constraints, and the need to have a low dissociation constant (K_{d}) for the triplex-forming molecules/target sequence. The molecules will have a base composition which is conducive to triple-helix formation and may also take into consideration the structural motifs for third strand binding. The most stable complexes are formed on polypurine elements, however, as discussed above this requirement is reduced by the inclusion of a tail sequence on the Watson-Crick binding portion.

Preferably, the triplex-forming molecules such as tcPNAs bind to or hybridize to the target sequence under conditions of high stringency and specificity. Most preferably, the triplex-forming molecules bind in a sequence-specific manner to the target sequence. Reaction conditions for in vitro triple helix formation of triplex-forming molecules to a nucleic acid sequence vary from molecule to molecule, depending on factors such as nucleotide length, the number of G:C and A:T base pairs, and the composition of the buffer utilized in the hybridization reaction.

Typically, triplex-helix forming molecules, such as PNAs, are substantially complementary to the target sequence. Preferably, both the Waston-Crick and Hoogsteen binding portions of the triplex forming molecules are substantially complementary to the target sequence.

Preferably, the triplex-forming molecules, such as PNAs, are between 6 and 50 nucleotides in length. The Watson-Crick portion should be 9 or more nucleobases in length, including the tail sequence. More preferably, the Watson-Crick binding portion is between about 9 and 30 nucleobases in length, including a tail sequence of between 0 and about 15 nucleobases. More preferably, the Watson-Crick binding portion is between about 10 and 25 nucleobases in length, including a tail sequence of between 0 and about 10 nucleobases. In the most preferred embodiment, the Watson-Crick binding portion is between 15 and 25 nucleobases in length, including a tail sequence of between 5 and 10 nucleobases. The Hoogsteen binding portion should be 6 or more nucleobases in length. Most preferably, the Hoogsteen binding portion is between about 6 and 15 nucleobases, inclusive.

PNA are typically designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobases, known as a “tail,” to the Watson-Crick binding portion of the triplex-forming molecules allows the Watson-Crick binding portion to bind/hybridize to the target strand outside the site of strand displacement. These additional bases reduce the requirement for the polypurine:polypyrimidine stretch in the target duplex and therefore increase the number of potential target sites. Triplex-forming oligonucleotides (TFOs) typically prefer a stretch polypurine:polypyrimidine to a form a triple helix. TFOs may require a stretch of at least 15 and preferably 30 or more nucleotides. Peptide nucleic acids require fewer purines to a form a triple helix, although at least 10 or preferably more may be needed. Peptide nucleic acids including a tail, also referred to as tail clamp PNAs, or tcPNAs, require even fewer purines to a form a triple helix. A triple helix may be formed with a target sequence containing fewer than 8 purines. Therefore, triplex-forming molecules including PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 16 to 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. Triplex-forming molecules can also be chemically modified using standard methods that are well known in the art.

3. Chemical Modifications to Triplex-Forming Molecules

Each nucleotide typically comprises a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

Under physiologic conditions, potassium levels are high, magnesium levels are low, and pH is neutral. These conditions are generally unfavorable to allow for effective binding of TFOs to duplex DNA. For example, high potassium promotes guanine (G)-quartet formation, which inhibits the activity of G-rich purine motif TFOs. Also, magnesium, which is present at low concentrations under physiologic conditions, supports third-strand binding by charge neutralization. Finally, neutral pH disfavors cytosine protonation, which is needed for pyrimidine motif third-strand binding. Target sequences with adjacent cytosines are particularly problematic. Triplex stability is greatly compromised by runs of cytosines, thought to be due to repulsion between the positive charge resulting from the N³ protonation or perhaps because of competition for protons by the adjacent cytosines.

Chemical modification of nucleobases, sugar moieties, and/or linkages comprising triplex-forming molecules may be useful to increase binding affinity of triplex forming molecules and/or triplex stability under physiologic conditions. Therefore, in some embodiments, the triplex-forming molecules including PNAs and other suitable oligonucleotides may include one or more modifications or substitutions to the nucleobases, sugars, or linkages to one or more of the nucleotides which make a triplex-forming molecule. As used herein “modified nucleotide” or “chemically modified nucleotide” defines a nucleotide that has a chemical modification of one or more of the heterocyclic base, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence. Modifications should not prevent, and preferably enhance, duplex invasion, strand displacement, and/or stabilize triplex formation as described above by increasing specificity or binding affinity of the triplex-forming molecules to the target site.

a. Heterocyclic Bases

The principal naturally-occurring nucleotides comprise uracil, thymine, cytosine, adenine and guanine as the heterocyclic bases. Triplex-forming molecules such as TFO's and PNAs can include chemical modifications to their nucleobase constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides comprising triplex-forming molecules such as TFOs and PNAs may be useful to increase binding affinity of triplex-forming molecules and/or triplex stability under physiologic conditions.

Chemical modifications of heterocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as TFOs and PNAs helps to stabilize triplex formation at neutral and/or physiological pH, especially in triplex-forming molecules with isolated cytosines. This is because the positive charge partially reduces the negative charge repulsion between the triplex-forming molecules and the target duplex, and allows for Hoogsteen binding.

b. Sugar Modifications

Triplex-forming molecules, particularly TFOs, may also contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

c. Internucleotide Linkages

The nucleotide subunits of the triplex-forming molecules such as TFOs and PNAs are connected by an internucleotide bond that refers to a chemical linkage between two nucleoside moieties.

Modifications to the phosphate backbone of triplex-forming oligonucleotides may increase the binding affinity of TFOs or stabilize the triplex formed between the TFO and the target duplex. Cationic modifications, including, but not limited to, diethyl-ethylenediamide (DEED) or dimethyl-aminopropylamine (DMAP) may be especially useful due to decrease electrostatic repulsion between TFO and duplex target phosphates. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds, which allow them to form PNA−DNA or PNA−RNA duplexes via Watson-Crick base pairing with high affinity and sequence-specificity. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications include peptide and amino acid variations and modifications. Thus, the backbone constituents of triplex forming molecules such as PNAs may be peptide linkages, or alternatively, they may be non-peptide peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

Linkage modifications used to generate triplex-forming molecules should not prevent the molecules from binding with high specificity to the target site and creating a triplex with the target duplex nucleic acid by displacing one strand of the target duplex and forming a clamp around the other strand of the target duplex.

Triplex-forming molecules such as PNAs may optionally include one or more terminal amino acids at either or both termini to increase stability, and/or affinity of the PNAs or modified nucleotides for DNA, or increase solubility of PNAs or modified nucleotides for duplex DNA. Commonly used positively charged moieties include the amino acids lysine and arginine, although other positively charged moieties may also be useful. For example, lysine and arginine residues can be added to a bis-PNA linker or can be added to the carboxy or the N-terminus of a PNA strand.

Triplex-forming molecules may further be modified to be end capped to prevent degradation using a 3′ propylamine group. Procedures for 3′ or 5′ capping oligonucleotides are well known in the art.

B. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the triplex-forming molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. For example, the K_{d} can be estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

C. Donor Oligonucleotides

The triplex-forming molecules can be administered alone or in combination with donor molecules. The donor molecules can be tethered, or non-tethered to the triplex-forming molecules. The tethered donor oligonucleotide can be tethered via a mixed sequence linker. Donor oligonucleotides are typically substantially homologous to a target sequence. Triplex-forming molecules can induce recombination of a donor oligonucleotide sequence up to several hundred base pairs away. It is preferred that the donor oligonucleotide sequence binding site is between 1 to 800 bases from the target of the triplex-forming molecules. More preferably the donor oligonucleotide sequence is between 25 to 75 bases from the target binding site of the triplex-forming molecules. Most preferably the donor oligonucleotide sequence is about 50 nucleotides from the target binding site of the triplex-forming molecules.

The donor sequence typically can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, donor molecules, and donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the olignucleotide donor is between 25 and 80 nucleobases. In a further embodiment, the non-tethered donor nucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences.

The donor oligonucleotides can contain a variety of mutations relative to the target sequence. Representative types of mutations include, but are not limited to, point mutations, deletions and insertions. Point mutations can cause missense or nonsense mutations. Deletions and insertions can result in frameshift mutations or deletions. These mutations may disrupt, reduce, stop, increase, improve, or otherwise alter the expression of the target gene. For example, it may be desirable to reduce or stop expression of an oncogene. Alternatively, it may be desirable to alter the polypeptide encoded by the target gene.

Compositions including triplex-forming molecules such as tcPNA may include one or more donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic bases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. Modifications to the donor oligonucleotide should not prevent the donor oligonucleotide from successfully recombining at the recombination target sequence in the presence of triplex-forming molecules.

In the most preferred embodiment, donor molecules are administered in combination with triplex-forming molecules, most preferably peptide nucleic acids. As shown in the examples below, donor molecules alone can induce recombination at the target site. Therefore, in some embodiments, donor molecules are administered without triplex forming molecules.

D. Methods for Determining Introduction of Alternative Sequence at the Target Site

Allele-specific PCR is a preferred method for determining if a recombination event has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing; analysis of mRNA transcribed from the target gene, for example, by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by, the target gene. For example, if the target gene encodes an enzyme, an assay designed to test enzyme function may be used.

E. Cell Targeting Moieties and Protein Transduction Domains

Formulations of the triplex-forming molecules embrace fusions of the triplex-forming molecules or modifications of the triplex-forming molecules, wherein the triplex-forming molecules are fused to another moiety or moieties. Such analogs may exhibit improved properties such as increased cell membrane permeability, activity and/or stability. Examples of moieties which may be linked or unlinked to the triplex-forming molecules, or donor oligonucleotides include, for example, targeting moieties which provide for the delivery of molecules or oligonucleotides to specific cells, e.g., antibodies to hematopoeitic stem cells, CD34⁺ cells, T cells or any other preferred cell type, as well as receptor and ligands expressed on the preferred cell type. Preferably, the moieties target hematopoeitic stem cells. Other moieties that may be provided with the triplex-forming molecules or oligonucleotides include protein transduction domains (PTDs), which are short basic peptide sequences present in many cellular and viral proteins that mediate translocation across cellular membranes. Exemplary protein transduction domains that are well-known in the art include the Antennapedia PTD and the TAT (transactivator of transcription) PTD, poly-arginine, poly-lysine or mixtures of arginine and lysine.

F. Additional Mutagenic Agents

The triplex-forming molecules can be used alone or in combination with other mutagenic agents. As used herein, two agents are said to be used in combination when the two agents are co-administered, or when the two agents are administered in a fashion so that both agents are present within the cell or blood simultaneously. In a preferred embodiment, the additional mutagenic agents are conjugated or linked to the triplex-forming molecule. Additional mutagenic agents that can be used in combination with triplex-forming molecules include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

G. Additional Prophylactic or Therapeutic Agents

The triplex-forming molecules can be used alone or in combination with other prophylactic or therapeutic agents. As used herein, two agents are said to be used in combination when the two agents are co-administered, or when the two agents are administered in a fashion so that both agents are present within the cell or serum simultaneously. Suitable additional prophylactic or therapeutic agents will be known to one of skill in the art and will depend on the parameters such as the patient and condition to be treated.

It may also be desirable to administer compositions containing triplex-forming molecules in combination with agents that further enhance the frequency of gene correction in cells. For example, the compositions can be administered in combination with a histone deacetylase (HDAC) inhibitor, such as suberoylanilide hydroxamic acid (SAHA), which has been found to promote increased levels of gene targeting in asynchronous cells. The nucleotide excision repair pathway is also known to facilitate triplex-forming molecule-mediated recombination. Therefore, the compositions can be administered in combination with an agent that enhances or increases the nucleotide excision repair pathway, for example, an agent that increases the expression, activity, or localization to the target site, of the endogenous damage recognition factor XPA. Compositions may also be administered in combination with a second active agent that enhances uptake or delivery of the triplex-forming molecules or the donor oligonucleotides. For example, the lysosomotropic agent chloroquine has been shown to enhance delivery of PNAs into cells (Abes, et al., J. Controll. Rel., 110:595-604 (2006).

### III. Targeting Molecules and Methods of Attachment to Microparticles

A. Targeting Molecules

Targeting molecules can be proteins, peptides, nucleic acid molecules, saccharides or polysaccharides that bind to a receptor or other molecule on the surface of a targeted cell. The degree of specificity can be modulated through the selection of the targeting molecule. For example, antibodies are very specific. These can be polyclonal, monoclonal, fragments, recombinant, or single chain, many of which are commercially available or readily obtained using standard techniques. Examples of molecules targeting extracellular matrix (“ECM”) include glycosaminoglycan (“GAG”) and collagen. In one embodiment, the external surface of polymer microparticles may be modified to enhance the ability of the microparticles to interact with selected cells or tissue, for example, wherein a fatty acid conjugate is inserted into the microparticle is preferred. In another embodiment, the outer surface of a polymer microparticle having a carboxy terminus may be linked to PAMPs that have a free amine terminus. The PAMP targets Toll-like Receptors (TLRs) on the surface of the cells or tissue, or signals the cells or tissue internally, thereby potentially increasing uptake. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the microparticle may be treated using a mannose amine, thereby mannosylating the outer surface of the microparticle. This treatment may cause the microparticle to bind to the target cell or tissue at a mannose receptor on the antigen presenting cell surface. Alternatively, surface conjugation with an immunoglobulin molecule containing an Fe portion (targeting Fe receptor), heat shock protein moiety (HSP receptor), phosphatidylserine (scavenger receptors), and lipopolysaccharide (LPS) are additional receptor targets on cells or tissue.

Lectins that can be covalently attached to microparticles to render them target specific to the mucin and mucosal cell layer include lectins isolated from Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to any microparticle may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microparticles with the appropriate chemistry, such as the fatty acid conjugates or CDI, and be expected to influence the binding of microparticles to the gut. For example, polyclonal antibodies raised against components of mucin or else intact mucin, when covalently coupled to microparticles, would provide for increased bioadhesion. Similarly, antibodies directed against specific cell surface receptors exposed on the lumenal surface of the intestinal tract would increase the residence time of beads, when coupled to microparticles using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the microparticles would decrease the surface tension of the bead-gut interface and increase the solubility of the bead in the mucin layer. The list of useful ligands includes sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, also increases bioadhesiveness. Using polyamino acids in the 15,000 to 50,000 kDa molecular weight range yields chains of 120 to 425 amino acid residues attached to the surface of the microparticles. The polyamino chains increase bioadhesion by means of chain entanglement in mucin strands as well as by increased carboxylic charge.

B. Methods of Attachment

Targeting molecules can be coupled directly to the polymer or to a material such as a fatty acid which is incorporated into the polymer. Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the microparticles, for example, during the emulsion preparation of microparticles by incorporation of stablizers with functional chemical groups, for example, whereby functional amphiphilic molecules are inserted into the particles during emulsion preparation. A second is post-particle preparation, by direct crosslinking particles and ligands with homo- or heterobifunctional crosslinkers. This second procedure may use a suitable chemistry and a crosslinker such as CDI, EDAC, glutaraldehyde, etc. or any other crosslinker that couples ligands to the particle surface via chemical modification of the particle surface after prepartion. This second class also includes a process whereby amphiphilic molecules such as fatty acids, lipids or functional stabilizers may be passively adsorbed and adhered to the particle surface, thereby introducing functional end groups for tethering to ligands.

In the preferred embodiment, the surface is modified to insert amphiphilic polymers or surfactants that match the polymer phase HLB or hydrophile-lipophile balance, as demonstrated in the following example. HLBs range from 1 to 15. Surfactants with a low HLB are more lipid loving and thus tend to make a water in oil emulsion while those with a high HLB are more hydrophilic and tend to make an oil in water emulsion. Fatty acids and lipids have a low HLB below 10. After conjugation with target group (such as hydrophilic avidin), HLB increases above 10. This conjugate is used in emulsion preparation. Any amphiphilic polymer with an HLB in the range 1-10, more preferably between 1 and 6, most preferably between 1 and up to 5, can be used. This includes all lipids, fatty acids and detergents.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfa NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method is useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

### III. Applications

Triplex-forming molecules such as TFO's and peptide nucleic acids (PNAs) are powerful gene therapy agents that can enhance recombination of short donor DNAs with genomic DNA, leading to targeted and specific correction of disease-causing genetic mutations. Therapeutic use of triplex-forming molecules has been limited, however, by challenges in intracellular delivery, particularly in clinically relevant targets such as hematopoietic stem and progenitor cells. For example, PNAs do not readily cross the cell membrane, so special delivery methods are required. The Amaxa nucleofection/electroporation system has been established as a superior method of DNA transfection for hematopoietic stem cells, however, it is somewhat toxic to cells, and cannot be used in vivo.

Microparticles and nanoparticles can be used to deliver triplex-forming oligonucleotides for a variety of in vitro and in vivo applications. Microparticles loaded with triplex forming nucleic acids and/or donor molecules facilitate delivery of the nucleic acids to the cell with low to no cytotoxicity. Once inside the cell, triplex-forming molecules bind/hybridize to a target sequence within or adjacent to a human gene, thereby displacing the polyprimidine strand, and forming a triplex structure and hybrid duplex with the polypurine strand. The binding of the triple-forming molecule to the target region stimulates mutations within or adjacent to the target region using cellular DNA synthesis, recombination, and repair mechanisms. In targeted recombination, a triplex forming molecule is administered to a cell in combination with a separate donor oligonucleotide fragment which minimally contains a sequence substantially complementary to the target region or a region adjacent to the target region, referred to herein as the donor fragment. The donor fragment can further contain nucleic acid sequences which are to be inserted within the target region. The co-administration of a triplex forming molecules with the fragment to be recombined increases the frequency of insertion of the donor fragment within the target region when compared to procedures which do not employ a triplex forming molecules.

If the target gene contains a mutation that is the cause of a genetic disorder, then the oligonucleotide is useful for mutagenic repair that restores the DNA sequence of the target gene to normal. If the target gene is a viral gene needed for viral survival or reproduction or an oncogene causing unregulated proliferation, such as in a cancer cell, then the mutagenic oligonucleotide is useful for causing a mutation that inactivates the gene to incapacitate or prevent reproduction of the virus or to terminate or reduce the uncontrolled proliferation of the cancer cell. The mutagenic oligonucleotide is also a useful anti-cancer agent for activating a repressor gene that has lost its ability to repress proliferation.

Compositions containing triplex-forming molecules are particularly useful as a molecular biology research tool to cause targeted mutagenesis. Targeted mutagenesis has been shown to be a very useful tool when employed to not only elucidate functions of genes and gene products, but alter known activities of genes and gene products as well. Targeted mutagenesis is also useful for targeting a normal gene and for the study of mechanisms such as DNA repair. Targeted mutagenesis of a specific gene in an animal oocyte, such as a mouse oocyte, provides a useful and powerful tool for genetic engineering for research and therapy and for generation of new strains of “transmutated” animals and plants for research and agriculture.

The induction of targeted mutatgenesis or recombination using microparticles to deliver triplex forming molecules and/or donor molecules may be used to correct a mutation in a target gene that is the cause of a genetic disorder. Alternatively, if the target gene is a viral gene needed for viral survival or reproduction or an oncogene causing unregulated proliferation, such as in a cancer cell, then the use of recombinagenic triplex-forming molecules, such as tcPNAs, should be useful for inducing a mutation or correcting the mutation, by homologous recombination, thereby inactivating the gene to incapacitate or prevent reproduction of the virus or to terminate or reduce the uncontrolled proliferation of the cancer cell.

The triplex-forming molecules can further be used to stimulate homologous recombination of an exogenously supplied, donor oligonucleotide, into a target region. Specifically, by activating cellular mechanisms involved in DNA synthesis, repair and recombination, the triplex-forming molecules can be used to increase the efficiency of targeted recombination.

In targeted recombination, triplex forming molecules are administered to a cell in combination with a separate donor fragment which minimally contains a sequence essentially complementary to the target region or a region adjacent to the target region, referred to herein as the donor fragment. As shown in the examples below, donor DNA administered alone is also recombinagenic. In some embodiments, the triplex-forming molecules and the donor oligonucleotides are loaded into the same nanoparticle. In some embodiments, the triplex-forming molecules and the donor oligonucleotides are loaded into separate microparticles. Separate microparticles may be delivered to a cell at the same time, or sequentially.

The triplex-forming molecules in conjunction with donor oligonucleotides can induce any of a range of mutations, including corrective mutations, in or adjacent to the target sequence. Representative types of mutations include, but are not limited to point mutations, deletions and insertions. Point mutations can cause missense or nonsense mutations. Deletions and insertions can result in frameshift mutations or deletions. The donor fragment can differ from the target sequence at the one or more base positions that are desired to be substituted, inserted, deleted, or otherwise altered. In some embodiments, the donor fragment contains nucleic acid sequences which are to be inserted within the target region. The co-administration of a triplex forming molecules with the fragment to be recombined increases the frequency of insertion of the donor fragment within the target region when compared to procedures which do not employ a triplex forming molecules.

The triplex-forming molecules in combination with the donor oligonucleotide induces site-specific mutations or alterations of the nucleic acid sequence within or adjacent to the target sequence. In one embodiment, the target sequence is preferably within or is adjacent to a portion of human beta-globin gene. Target sequences can be within the coding DNA sequence of the gene or within introns. Target sequences can also be within DNA sequences which regulate expression of the target gene, including promoter or enhancer sequences.

The examples demonstrate efficient and non-toxic PNA-mediated recombination in human CD34⁺ cells using poly(lactic-co-glycolic acid) (PLGA) nanoparticles for intracellular oligonucleotide delivery. As shown below, treatment of progenitor cells with nanoparticles loaded with PNAs and DNAs targeting the beta-globin locus led to levels of site-specific modification in the range of 0.5-1% in a single treatment, without detectable loss in cell viability, resulting in a 60-fold increase in modified and viable cells as compared to nucleofection. The differentiation capacity of the progenitor cells treated with nanoparticles did not change relative to untreated progenitor cells, indicating that nanoparticles are safe and minimally disruptive delivery vectors for PNAs and DNAs to mediate gene modification in human primary cells.

As noted above, the term “microparticle” includes “nanoparticles” unless otherwise stated. In the most preferred embodiments, the microparticles are nanoparticles. The preferred size of microparticles loaded with triplex-forming molecules and optionally a donor DNA, is between about 10 nm and 1000 nm, preferably about 50 nm and 500 nm, most preferably between about 100 nm and 200 nm. The examples below illustrate particle having sizes 156+/−49 nm for blank particles, 150+/−42 nm for DNA particles, 132+/−31 nm for PNA particles, and 156+/−51 nm for PNA−DNA particles.

Loading of the nucleic acids into the microparticles can typically range from about 0.01% to about 5% w/w. It is believed that loading as little as 0.01% w/w of nucleic acids into microparticles will be sufficient for targeted recombination in cells. Loading of percentages greater than 5% is also contemplated. Alternatively, as shown the examples below, nucleic acids can be expressed as moles of nucleic acid per unit mass of microparticles. For example the loading range for nucleic acids is from about 0.1 nmole to 10 nmole of nucleic acid per milligram of microparticles, though higher and lower amounts are also contemplated. Preferably, the loading range for nucleic acids is about 0.25 nmole to 2.5 nmole In the most preferred embodiment, the loading ratio is about 1 mole nucleic acid per milligram microparticle, for example 1 nmole nucleic acid per milligram PLGA.

The examples below show that an equimolar ratio (i.e. 1:1) of DNA (donor oligonucleotide) and PNA (triplex-forming molecule) results in a DNA:PNA ratio of approximately 1:2 loaded into the microparticles. It is believed that the starting ratio of DNA:PNA can be manipulated to adjust the ratio of DNA:PNA loaded into the nanoparticle.

Preferred dosages will vary depending on the application and the subject to be treated, and can be determined using standard assays that are known in the art. Preferred dosages for in vitro and ex vivo applications can range from about 0.1 mg/ml to about 10 mg/ml, preferable between about 0.2 mg/ml and 5 mg/ml, most preferably about 2 mg/ml. Alternatively, dosages can be expressed as the number of particles/cell. For example, preferred dosages may range from about 1×10⁴ particles/cell to 1×10⁷ particles/cell, preferably between about 1×10⁵ particles/cell and 1×10⁶ particles/cell. In vivo dosages will also vary depending on the disorder or disease, the subject to be treated, and the method of administration. For example, in vivo dosages by systemic injection can range from about 0.005 gram particles/gram weight of animal to 0.5 gram particles/gram weight of animal.

A. Methods of Use as a Molecular Research Tool

For in vitro research studies, microparticles containing the triplex-forming molecules is added directly to a solution containing the DNA molecules of interest in accordance with methods well known to those skilled in the art and described in more detail in the examples below.

In vivo research studies are conducted by treating cells with the microparticles containing triplex-forming molecules and optionally one or more donor oligonuleotides in a solution such as growth media for a sufficient amount of time for entry of the triplex-forming molecules into the cells for triplex formation with a target duplex sequence. The target duplex sequence may be episomal DNA, such as nonintegrated plasmid DNA. The target duplex sequence may also be exogenous DNA, such as plasmid DNA or DNA from a viral construct, which has been integrated into the cell's chromosomes. The target duplex sequence may also be a sequence endogenous to the cell. The transfected cells may be in suspension or in a monolayer attached to a solid phase, or may be cells within a tissue wherein the triplex-forming molecules are in the extracellular fluid.

B. Methods of Use for Treatment of Medical Conditions

The relevance of DNA repair and mediated recombination as gene therapy is apparent when studied in the context of human genetic diseases such as cystic fibrosis, hemophelia, globinopathies such as sickle cell anemia and beta-thalassemia, and lysosome storage diseases such as Hurler's syndrome or Gaucher's disease. If the target gene contains a mutation that is the cause of a genetic disorder, then the oligonucleotide is useful for mutagenic repair that may restore the DNA sequence of the target gene to normal.

Targeted DNA repair and recombination induced by triplex-forming molecules and/or donor molecules delivered using microparticles is especially useful to treat genetic deficiencies, disorders and diseases caused by mutations in single genes. Triplex-forming molecules are also especially useful to correct genetic deficiencies, disorders and diseases caused by point mutations.

Worldwide, globinopathies account for significant morbidity and mortality. Over 1,200 different known genetic mutations affect the DNA sequence of the human alpha-like (HBZ, HBA2, HBA1, and HBQ1) and beta-like (HBE1, HBG1, HBD, and HBB) globin genes. Two of the more prevalent and well-studied globinopathies are sickle cell anemia and β-thalassemia. Substitution of valine for glutamic acid at position 6 of the β-globin chain in patients with sickle cell anemia predisposes to hemoglobin polymerization, leading to sickle cell rigidity and vasoocclusion with resulting tissue and organ damage. In patients with β-thalassemia, a variety of mutational mechanisms results in reduced synthesis of β-globin leading to accumulation of aggregates of unpaired, insoluble α-chains that cause ineffective erythropoiesis, accelerated red cell destruction, and severe anemia. Methods for targeting the beta-globin gene are described in the examples below, in U.S. Application No. 2007/0219122 and PCT/US2010/031888.

All together, globinopathies represent the most common single-gene disorders in man. Triplex forming molecules are particularly well suited to treat globinopathies, as they are single gene disorders caused by point mutations. The Example that follows demonstrates that triplex-forming molecules, such as tcPNAs are effective at binding to the human β-globin both in vitro and in living cells. The Example further demonstrates, the tcPNAs targeted to specific target sites in the human β-globin gene and effectively induce repair of known mutations when co-administered with appropriate donor oligonucleotides.

If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation.

The oligonucleotide is useful as an antiviral agent when the oligonucleotide is specific for a portion of a viral genome necessary for proper proliferation or function of the virus.

The disclosed compositions are also useful for targeting other gene disorders which are known in the art. As described in the examples below, microparticles loaded with triplex forming molecules can be used for targeted correction of the CCR5 gene, as described in WO 2008/086529.

1. Ex Vivo Gene Therapy for Treating or Preventing Genetic Disorders

In one embodiment, ex vivo gene therapy of cells is used for the treatment of a genetic disorder in a subject. For ex vivo gene therapy cells are isolated from a subject and contacted ex vivo with the compositions to produce cells containing mutations in or adjacent to genes. In a preferred embodiment, the cells are isolated from the subject to be treated or from a syngenic host. Target cells are removed from a subject prior to contacting with triplex-forming molecules and donor oligonucleotides. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with sickle cell anemia, the beta-globin gene altered or repaired ex-vivo using the disclosed compositions and methods, and the cells reintroduced back into the patient as a treatment or a cure.

Such stem cells can be isolated and enriched by one of skill in the art.

Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 has been deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium comprising cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and/or donor oligonucleotides loaded into microparticles in amounts effective to cause the desired mutations in or adjacent to genes in need of repair or alteration, for example the human beta-globin gene. These cells are referred to herein as modified cells.

The modified cells can be maintained or expanded in culture prior to administration to a subject. Culture conditions are generally known in the art depending on the cell type. Conditions for the maintenance of CD34⁺ in particular have been well studied, and several suitable methods are available. In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes a period week to months.

A high percentage of engraftment of modified hematopoietic stem cells cells is not envisioned to be necessary to achieve significant prophylactic or therapeutic effect. It is expected that the engrafted cells will expand over time following engraftment to increase the percentage of modified cells. In some embodiments, the modified cells have a corrected beta-globin gene. Therefore, in a subject with sickle cell anemia or other globinopathies, the modified cells are expected to improve or cure the condition. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic. Nevertheless, allogeneic cell transplants are also envisioned, and allogeneic bone marrow transplants are carried out routinely. Allogeneic cell transplantation can be offered to those patients who lack an appropriate sibling donor by using bone marrow from antigenically matched, genetically unrelated donors (identified through a national registry), or by using hematopoietic progenitor or stem-cells obtained or derived from a genetically related sibling or parent whose transplantation antigens differ by one to three of six human leukocyte antigens from those of the patient.

2. In Vivo Gene Therapy

In another embodiment, the triplex-forming molecules are administered directly to a subject in need of gene alteration. As used herein the terms “drug” and “bioactive agent” includes triplex-forming molecules and optionally DNA donor. Therefore, a microparticle is loaded with “drug” or “bioactive agent” if it is loaded with triplex-forming molecules or DNA donor alone or in combination.

C. Methods of Administration

Routes of administration can include any relevant medical, clinical, surgical, procedural, and/or parenteral route of administration including, but not limited to, intravenous, intraarterial, intramuscular, intraperitoneal, subcutaneous, intradermal, infusion, subconjunctive, and intracatheter (e.g., aurologic delivery), as well as administration via external scopic techniques such as, for example, arthroscopic or endoscopic techniques. The compositions can be administered to specific locations (e.g., local delivery).

In one embodiment, the microparticle composition is in a liquid suspending medium, which is also called an injection vehicle or fluid or diluent prior to administration. These suspensions are typically heterogeneous systems containing the solid, essentially insoluble dispersed material (the microparticle composition) suspended or disbursed in a liquid phase (the injection vehicle). The injection vehicle is typically sterile, stable, and capable of being delivered through a needle without clogging or otherwise blocking the delivery of the microparticle suspension.

The present invention will be further understood by reference to the following non-limiting examples.

### EXAMPLES

### Example 1

### Preparation of DNA, PNA, and DNA/PNA-loaded Nanoparticles

### Materials and Methods

Oligonucleotides

The following oligonucleotides were used throughout the examples below. Bis-PNA-194 with an 8-amino-2,6-dioxaoctanoic acid linker was purchased from Bio-Synthesis (Lewisville Tex.) and purified by HPLC. Bis-PNA-194 has six terminal lysines at the N terminus. Donor oligonucleotides 50 nt in length were synthesized by Midland Certified Reagent (Midland Tex.), 5′- and 3′-end protected by three phosphorothioate internucleoside linkages at each end and purified by reversed phase-HPLC. The donor DNAs are homologous to the human beta globin gene, except for a 6 nucleotide change centered at the junction of exon 2 and intron 2. This 6 nt sequence change enables reliable detection of the genomic sequence modification by allele-specific PCR.

bis-PNA IVS2-194: Lys-Lys-Lys-Lys-Lys-Lys-JJT JTT JTT OOO TTC TTC TCC (SEQ ID NO:1), where J=pseudoisocytosine, O=flexible 8-amino-3,6-dioxaoctanoic acid, 6-aminohexanoic acid monomers, T=thymine, C=cytosine.

Labeled control PNA for measuring loading: Fluorescein-oo-Lys-TATGACATGAACT-Lys-Lys-Lys-Lys (SEQ ID NO:2)

β-globin donor DNA: 5′-AAA CAT CAA GGG TCC CAT AGG TCT ATT CTG AAG TTC TCA GGA TCC ACG TG-3′ (SEQ ID NO:3), where the mutated base pairs are underlined.

Nanoparticle Formulation

Poly(lactic-co-glycolic acid) (PLGA) nanoparticles were formulated by a double-emulsion solvent evaporation technique [Fahmy, et al (2005) Biomaterials 26: 5727-5736]. Nucleic acid and spermidine amounts were chosen based on optimized amounts used by Woodrow et al for siRNA encapsulation [Woodrow, et al (2009) Nat Mater 8: 526-533]. PNA, PNA and DNA, or DNA and spermidine were dissolved in 61.6 μL DNAse/RNAse free H₂O. PNA batches had 80 nmoles PNA, PNA−DNA batches had 40 nmoles of each, and DNA batches had 80 nmoles DNA and 1.08 mg spermidine. The encapsulant in H₂O was then added dropwise to a polymer solution of 80 mg 50:50 ester-terminated PLGA dissolved in 800 μL dichloromethane (DCM), then sonicated to form the first emulsion. This emulsion was then added dropwise to 1.6 mL 5% polyvinyl alcohol (PVA), then sonicated to form the second emulsion. This mixture was poured into 20 mL 0.3% PVA, and stirred at room temperature for 3 hours. Nanoparticles were then collected and washed with H₂O three times by centrifugation, then resuspended in H₂O, frozen at −80° C., and lyophilized. Particles were stored at −20° C. following lyophilization.

Lower spermidine concentrations for DNA only particles were also attempted but did not yield as high encapsulation efficiencies and release. Particles with 80 nmoles DNA and 67.6 μg spermidine or no spermidine had loadings of 272+/−52 pmoles/mg and 250+/−60 pmoles/mg loading respectively.

Coumarin 6 (C6) particles were formulated by single emulsion technique. C6 (Ex=460 nm, Em=500 nm; Sigma) was dissolved in DCM at 20 mg/mL, then added to PLGA dissolved in DCM (200 mg/2 mL). This mixture was added dropwise to 4 mL 5% PVA, sonicated, then poured into 0.3% PVA and stirred for 3 hours, then washed by centrifugation, frozen, and lyophilized.

Nanoparticle Characterization

To determine the amount of nucleic acid encapsulated in the nanoparticles, aqueous phase extraction was performed as described by Woodrow, et al., 2009. Briefly, 4 to 6 mg of nanoparticles from each batch were dissolved in 0.5 mL DCM at room temperature for 1 hour. 0.5 mL 10 mM Tris-HCl/1 mM EDTA pH 7.4 (TE buffer) was added to the DCM, vortexed 1-2 min, then centrifuged at 12000 RPM for 5 min at 4° C. The aqueous phase was then removed, and the procedure was repeated with another 0.5 mL TE buffer, for a total extraction volume of 1 mL. Absorbances at 260 nm were then measured with a Nanodrop 8000 (Thermo Scientific, Waltham, Mass.), and compared to PNA or DNA standards. To determine percent PNA and DNA in mixed particles, fluorescein labeled PNA was used, and emission from extract compared to standards. Release of nucleic acid was also analyzed by incubating 4 to 6 mg particles in 600 μL PBS in 37° C. shaker, spinning down and removing supernatant to measure absorbance 260 nm.

Scanning Electron Microscopy

Samples were coated with 25 nm-thick gold using a sputter coater. Images were analyzed using Image software (National Institute of Health), with greater than 500 particles analyzed per batch to determine size distribution. Briefly, brightness, contrast, and threshold were adjusted to enhance particle outlines, then Imagers “Analyze Particles” function was used to calculate the area of each particle.

### Results

PLGA nanoparticles loaded with DNA, PNA, or PNA and DNA were formulated by a double-emulsion solvent evaporation technique. All particle batches showed similar size around 150 nm, with uniform spherical morphologies, and were loaded densely with nucleic acid, as evident by scanning electron microscopy. “Blank” nanoparticles were loaded with phosphate buffered saline. Average particle diameter and standard deviation were found to be Blank:156±49 nm; DNA: 150±42 nm; PNA: 132±31 nm; DNA+PNA: 156±51 nm.

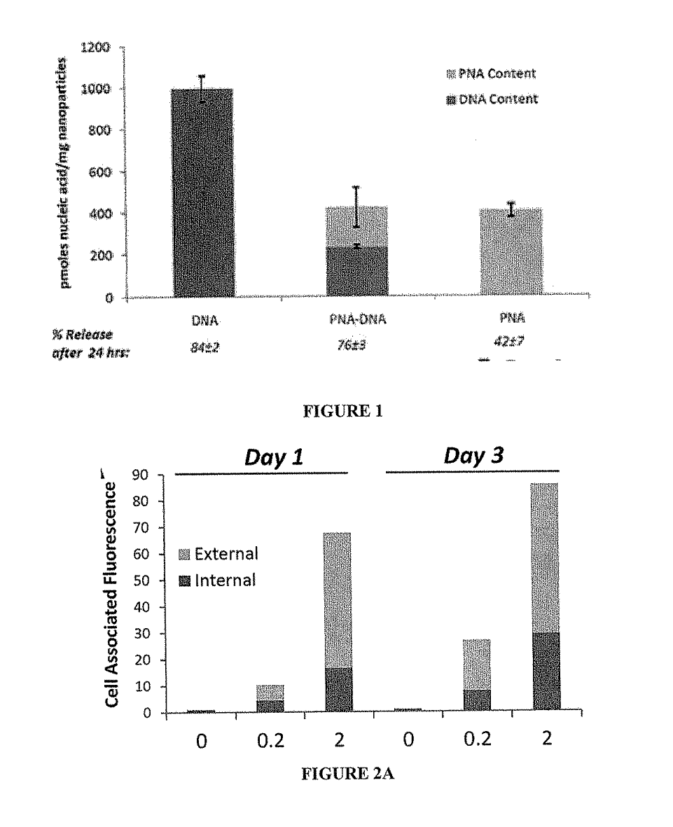

As shown in FIG. 1, nanoparticles can be densely loaded with DNA and/or PNA. Batches were loaded with 1 nmole DNA +13.5 μg spermidine/mg PLGA (“DNA”), 0.5 nmole PNA +0.5 nmole DNA/mg PLGA (“PNA−DNA”), or 1 nmole PNA/mg PLGA (“PNA”). Spermidine is not needed to package PNA, or DNA and PNA in combination, when the PNA sequence includes terminal lysines. Without being bound by theory, it is believed that terminal lysines on the PNA oligonucleotides provide positive charge that enhances loading of the nucleic acid(s) into the nanoparticle. It is further believed that PNA with terminal lysines provides a counter-ion for DNA loading in the PNA/DNA mixtures.

Loading of PNA and DNA per mg of nanoparticles is given +/− standard deviation, n=4 for each batch. As also shown in FIG. 1, a high percent release of encapsulant was observed after 24 hours when particles were incubated in PBS. Percent release of nucleic acid after 24 hours incubation at 37° C. is shown below the loading data (FIG. 1).

In summary, nanoparticles encapsulating donor DNA, PNA alone, or 50/50 mixtures of PNA and DNA at high levels were created.

### Example 2

### Nanoparticle Uptake by Human CD34+ Cells

### Materials and Methods

Primary Human CD34⁺ Cells

Human CD34⁺ cells were obtained from the Yale Center of Excellence in Molecular Hematology (Yale University, New Haven, Conn.) from granulocyte colony-stimulating factor (G-CSF)-mobilized peripheral blood of normal healthy donors. Cells were received frozen, then pooled, thawed, and maintained in StemSpan Serum-Free Expansion Medium (SFEM) with StemSpan CC100 cytokine mixture (Stemcell Technologies, Vancouver, Canada) (expansion medium). Antibiotics were added 24 hours after thawing (Primocin, Amaxa, Walkersville, Md.). After treatment with nanoparticles or by nucleofection, cells were maintained in expansion media or differentiation media. Erythroid differentiation media consisted of 2 U/ml erythropoietin and 50 ng/ml insulin-like growth factor 1 in StemSpan Serum-Free Expansion Medium. Neutrophil differentiation media consisted of 50 ng/ml SCF, 100 ng/ml Flt-3L, 5 ng/ml IL-3, 5 ng/ml granulocyte macrophage colony stimulating factor, and 30 ng/ml granulocyte colony stimulating factor in StemSpan SFEM for the first 5 days after treatment, followed by 5 ng/mL IL-3 and 30 ng/ml GCSF in SFEM for the next 5 days, thereafter 30 ng/mL GCSF in SFEM.

Cell counts were performed with a Nexcelcom Cellometer Auto T4 (Bioscience, Lawrence, Mass.) using trypan blue staining to identify dead cells.

Cell Transfection

24 hours after thawing, cells were nucleofected or particles were added. The treatment day is referred to as Day 0. Nucleofections (Amaxa Human CD34+ Nucleofection Kit, Lonza Group Ltd, Basel Switzerland) were performed as described by Chin, J Y, et al. (2008). Proc Natl Acad Sci U S A 105: 13514-13519. Approximately 1×10⁶ cells were nucleofected in 100 μL complete media with 0.2 nmoles DNA or 0.2 moles DNA plus 0.8 nmoles PNA (corresponding to concentrations of 2 μM DNA or 2 μM DNA/8 μM PNA or 1.2×10⁸ molecules of donor DNA per cell). Particles were resuspended in the StemSpan culture media with cytokines and added directly to 1×10⁶ cells at dosages indicated in the results (0.5 mg/mL corresponding to 1.2×10⁸ molecules of donor DNA per cell for DNA only, 0.24×10⁸ for PNA−DNA combined particles). For differentiation, after one day of treatment cells were pelleted and resuspended in fresh media containing the indicated cytokines.

“Mock nucleofected” cells were put through the nucleofection procedure but without DNA or PNA. “Low dose” PNA−DNA nanoparticles (nps) were 0.5 mg/mL. Low dose PNA+DNA nps is 0.25 mg/mL PNA nps+0.25 mg/mL DNA nps. Low dose DNA nps is 0.25 mg/mL nps. Low dose blank nps is 0.5 mg/mL. Low dose treatments were performed in triplicate. “Medium dose” nanoparticle treatments were all 2× that of low dose, and “high dose” nanoparticle treatments were all 4× that of low dose. Nanoparticle dosages were chosen based on the coumarin 6 uptake studies. Nucleofection doses were based on optimization described in Chin 2008.

FACS for Cell Surface Marker Expression and Particle Uptake

After 20 minute incubation on ice in 100 μL, PBS/1% FBS with 0.5 μL human CD16 (nonspecific block, Cat#555404), cells were incubated in 100 μL PBS/1% FBS with 1:50 dilution of antibody. Antibodies used were CD34 PE (Cat#550619), Glycophorin A (Cat#555570) PE, CD15 FITC (Cat#555401), IgG PE (Cat#555787), and IgM FITC (Cat#555583) Becton Dickenson/Pharmingen, Franklin Lakes, N.J.). After washing twice with PBS/1% FBS, cells were resuspended and analyzed using a FACSCalibur flow cytometer. Data was analyzed using FloJo software. Thresholds for positive signal were set by the Ig-isotype cell-stained controls. GlyA+ percentages for particles, blank particles, and untreated were 37, 36, and 39.1 at Day 4, 38, 15, and 30 at Day 8, and 54, 64, and 54.2 at Day 15. CD 15+ percentages for particles, blank particles, and untreated were 5, 2, and 0.19 at Day 4, 32, 23, and 28 at day 8, and 27, 23, and 21 at Day 15. All staining procedures were performed on ice or at 4° C.

In experiments using coumarin 6 labeled nanoparticles, FACS was used to determine particle uptake, using Trypan Blue to quench extracellular fluorescence [Van Amersfoortet al (1994) Cytometry 17: 294-301]. After treatment with particles, cells were harvested, resuspended in 1 mL PBS/1% FBS, and 1 mL of 600 μg/mL Trypan Blue was added. After 2 minutes incubation, cells spun down and resuspended in 1 mL PBS/1% FES, then analyzed by FACSCalibur.

Confocal Microscopy

Confocal images of cells treated with 2 mg/mL coumarin 6 (C6) nanoparticles were taken after 1 and 3 days of treatment. Approximately 100,000 cells were taken from each sample, washed twice by centrifugation in 1 mL PBS (2000 RPM, 5 min), then resuspended in 50 μL PBS plus 50 μL FBS. The samples were then spun onto slides using a Cytospin 3 machine, at 400 RPM, 5 min. Slides were placed in petri dishes for staining. Cells were fixed with 2 mL 4% paraformaldehyde at 37° C. for 15 min. After washing 3 times for 5 min with 10 mL PBS, cells were permeabilized with 5 mL 0.1% Triton-X-100 in PBS for 7 min at room temperature. After another 3 washes with 10 mL PBS, slides were incubated with 1 mL 1:10 Texas Red Phalloidin (Invitrogen) in PBS with 1% bovine serum albumin. After another 3 washes with PBS and one wash with H₂O, slides were air dried. 20 μL vectashield hard set mounting media with DAPI was added to each sample (Vectorlabs), coverslipped, and then allowed to harden at 15 min room temp, then 4° C. overnight.

Slides were then imaged with a Leica TCS SP5 Spectral Confocal Microscope. z-stack series were taken with 8 to 12 images per stack. The same fluorescence compensation settings were used for both C6 treated and untreated cells. Post-imaging, overall brightness and contrast of images were increased using ImageJ.

### Results

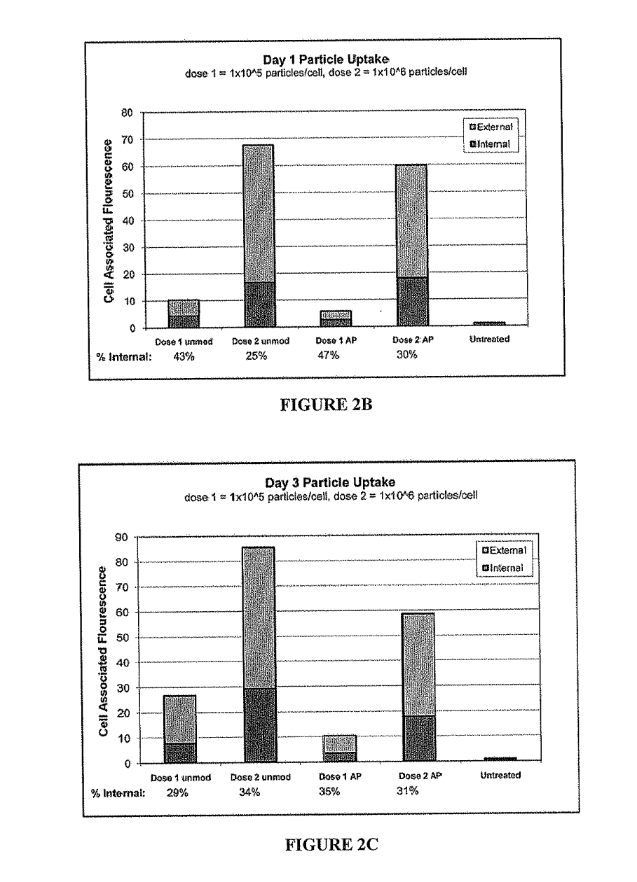

PLGA nanoparticles readily associate with and are taken up by hematopoietic cells. The fluorescent dye coumarin 6 (C6) was used to track cellular uptake of nanoparticles (FIGS. 2A-D) because C6 is not released from the particles after formulation. C6 nanoparticles were added to CD34⁺ hematopoietic progenitors obtained from the peripheral blood of healthy human donors, and cell-based fluorescence was measured by fluorescence activated cell sorting (FACS) after 1 and 3 days. CD34⁺ cells were plated overnight, then coumarin 6 loaded nanoparticles (206+/−73 nm) were added at the indicated concentrations shown in FIG. 2A. Uptake was measured by FACs (arbitrary fluorescence units) at Day 1 and Day 3 of treatment.

Cell association and uptake of nanoparticles with an antennapedia peptide was also investigated. Antennapedia peptide is a cell-penetrating peptide which, without being bound by theory, may improve intracellular delivery of the nanoparticles. As shown in of FIGS. 2B, 2C, and 2D show CD34+ cells internalize nanoparticles with or without antennapedia peptide (“AP”), at two doses (1×10⁵ particles/cell and 1×10⁶ particles/cell) as shown by FACS analysis at day 1, day 3, and day 5 respectively.

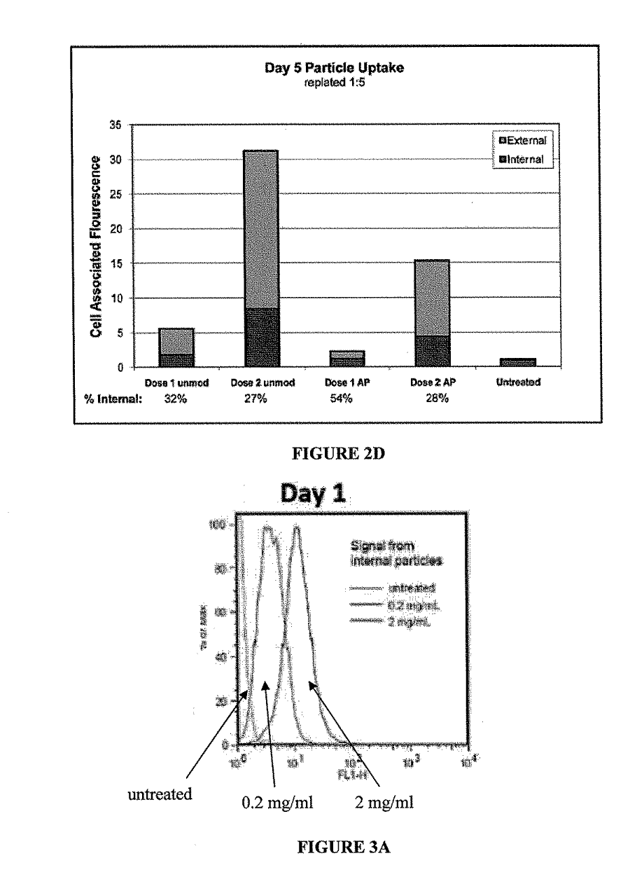

Trypan blue was used to quench externally attached particles to differentiate between signal from cell-associated (external) and internalized particles. Trypan blue can quench any fluorescence from external particles. High fluorescence signals were detected for both external (no quenching) and internalized particles. Cells are 98% CD34⁺ at Day 1. As shown in the histogram in FIG. 3A, using untreated as baseline, 80.9% of cells treated with 0.2 mg/mL coumarin 6 showed internalization, and 99.1% of cells treated with 2 mg/mL showed internalization at Day 1. FIG. 3B shows a similar assay featuring nanoparticles with or without antennapedia peptide (“AP”), at two doses (1×10⁵ particles/cell and 1×10⁶ particles/cell).

The findings shown in FIGS. 2A, 2B, 2C, 2D, 3A and 3B indicate that (1) the particles associated well with CD34 cells, (2) a large number of particles stick to the plasma membrane, (3) a significant percentage of these particles are internalized, and (4) this percentage is high enough that nearly all cells have at significant detectable amount of internalized particles when treated at 2 mg/mL.

Results from FACS were confirmed qualitatively with confocal microscopy. Cells were stained with Texas Red Phalloidin and DAPI (Blue). The low cytoplasm to nucleus ratio of CD34 cells makes internalization difficult to visualize, but images of mid-cell slices confirm that the particles are in the intracellular space. Fluorescence is not seen in the nucleus because particles are confined to the cytoplasm and coumarin 6 does not diffuse out of particles. These initial studies confirm that particles accumulate in the cytoplasm of CD34+ cells. This is consistent with previous studies showing localization of nanoparticles in several cytoplasmic compartments in epithelial cells [Cartiera, et al (2009) Biomaterials 30: 2790-2798].

In summary, in addition to high loading levels (Example 1), high percent release of particle contents after 24 hours was found. This is an important property if PNA and DNA are to be functional once the particles are internalized. Using C6 as a marker, it was shown that PLGA nanoparticles associate with and are internalized by human CD34⁺ cells at substantial levels.

### Example 3

### Cell Viability

Next, human CD34⁺ cells were treated with nanoparticles loaded with DNA and PNA and the cells examined for viability as compared to cells treated with DNA and PNA through optimized nucleofections. One day after CD34⁺ cells were thawed, nucleofections were performed or nanoparticles were added directly to cells. All treatment groups began with cells from identical populations of CD34⁺ cells from the same pool. All treatment groups began with an identical number of cells for each experiment. Nucleofection of the CD34⁺ cells was performed as described by Chin et al. 2008, and cells were spun down and resuspended in 2 mL culture medium. Nanoparticles were resuspended in culture medium and were added directly to cell cultures at dosages ranging from 2 to 0.25 mg/mL, in a total volume of 2 mL. Particles with DNA alone, both PNA and DNA (PNA−DNA), or separately loaded with PNA and DNA (PNA+DNA) were added to cells. “Untreated” cells were maintained in regular media without additional manipulation. Cell counts were performed using trypan blue to distinguish between live and dead cells.

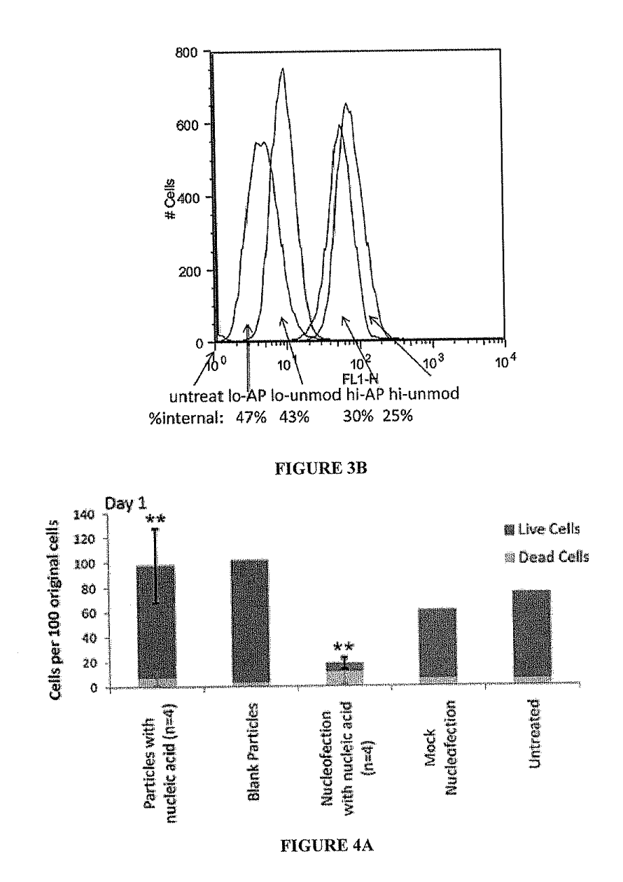

Cell survival and CD34 expression for nanoparticle-treated cells were found to be nearly identical to untreated cells (FIGS. 4A and 4B). In contrast, cell survival was substantially lower for nucleofected cells, and CD34⁺ expression was also reduced. Cell counts and FACS for CD34 expression were performed at several time points to assess toxicity of these treatments. The data represent averages for particles with nucleic acid (PNA, PNA−DNA, or PNA+DNA particles at all doses indicated above), and for nucleofection (PNA or PNA+DNA). For each experiment, an identical cell population and cell number were treated for each treatment group. Cell counts performed 1 (FIG. 4A) and 3 (FIG. 4B) days post-treatment with trypan blue staining was performed to identify dead cells. Counts are normalized to original cell platings. Error bars for live and dead cells give standard deviation where available. **p=0.01, ***p=5×10⁻¹².

Cell retention and survival for particle treated cells was similar to or better than untreated controls, while nucleofected cells had significantly lower total cell numbers and percent live cells, at both 1 (FIG. 4A) and 3 (FIG. 4B) days. In addition, cell retention/survival for blank particles was higher than cell survival with mock nucleofection at both 1 and 3 days post-treatment.

FIGS. 4C, 4D, and 4E show a similar assay featuring nanoparticles with or without antennapedia peptide (“AP”), at two doses (1×10⁵ particles/cell and 1×10⁶ particles/cell) on days 1, 3, and 5 respectively.

As shown in Table 1 below, starting with a sorted CD34⁺ population, cells remained 96-98% CD34⁺ for all treatment groups after 1 day of treatment. CD34 expression was uniform across treatment groups through day 3, although expression was lower for nucleofected cells at day 7. Data is given as % CD34⁺ with standard deviation. Day 1: data for only low dose particle treatments is available. Day 3 and 7: data for all doses available. *p=0.0002.

TABLE 1
CD34 expression of treated cells in non-differentiating
expansion media

| | Particles | | | | |
|---|---|---|---|---|---|
| | with | | | | |
| | nucleic | | Nucleofection | | |
| % | acid, all | Blank | with nucleic | Mock | |
| CD34+ | doses | Particles | acid, all doses | nucleofection | Untreated |
| Day 1 | 98.4 ± 0.6 | 97.9 | 96 ± 4 | 98.2 | 97.9 |
| | (n = 4) | | (n = 4) | | |
| Day 3 | 86 ± 4 | 84 ± 2 | 80 ± 15 | 88 | 84 ± 5 |
| | (n = 18) | (n = 5) | (n = 14) | (n = 2) | (n = 3) |
| Day 7 | 17 ± 1 | 16 ± 2 | 11 ± 3 | 15 | 15.3 ± 0.3 |
| | (n = 18)* | (n = 5) | (n = 14)* | (n = 2) | (n = 3) |

### Example 4

### Nanoparticles Facilitate Genomic Medication in Human Cells

### Materials and Methods

Allele-Specific Genomic PCR

Genomic DNA was harvested from CD34⁺-derived cells and purified using the Wizard Genomic DNA Purification kit (Promega, Madison Wis.). Equal amounts of genomic DNA were subjected to allele-specific PCR, in which the 3′ end of the forward primer corresponds to the wild-type or mutated sequence as introduced by the donor DNA. The PCR conditions are as follows, where the annealing temperature varies with primer set: 94° for 2 minutes; 35 cycles of 94° for 30 seconds, annealing for 30 seconds, and 72° for 1 minute; followed by 72° for 5 minutes. The annealing temperatures vary from 60° to 64°, and were determined empirically for each primer pair. Primer sequences available on request. As an experimental control, PCR was also performed on samples containing untreated (i.e. wild-type) CD34⁺ genomic DNA, spiked with DNA donor oligonucleotide immediately prior to the start of the PCR thermocycling reaction.

Genomic DNA Gel Purification

Genomic DNA from cells treated with particles containing both PNA and DNA, or nucleofected concurrently with bis-PNA and donor DNA, was harvested as above using the Wizard Genomic Purification Kit (Promega), and then electrophoresed in a 1% low melting point agarose gel in TAE, to separate genomic DNA from possible residual PNA and/or DNA oligonucleotide. The high molecular weight species, representing genomic DNA, was cut from the agarose gel and extracted using the Wizard SV Gel and PCR Clean-Up System (Promega) according to manufacturer's instructions. A subsequent allele-specific PCR was performed on this gel-purified genomic DNA to exclude the possibility of theoretical PCR artifact arising from the presence of residual oligonucleotide.

### Results

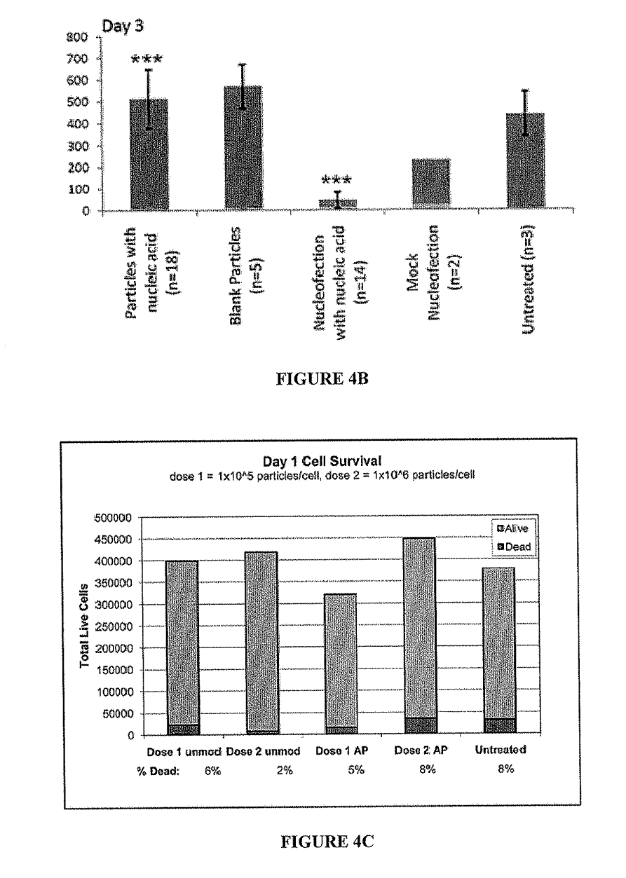

Next the ability of the oligonucleotide cargo within the nanoparticles to stimulate genomic recombination to modify the IVS2-1 splice site within the beta-globin gene was tested, as in Chin et al 2008. FIG. 5A is a schematic showing bis-PNA stand-displacement and triplex formation at a target site on a DNA duplex. FIG. 5B is a schematic of the PNA−DNA model system used to investigate nucleic acid loaded nanoparticle-mediated stimulation of genomic recombination to modify the IVS2-1 splice site within the beta-globin gene. The PNA binds within intron 2 of the endogenous β-globin locus. The single-stranded, 50-mer donor DNA molecule is homologous to the beta-globin gene, except for a 6 nucleotide sequence change, designed for gene modification at the exon 2/intron 2 boundary that produces a thalassemia-causing mutation. Allele specific PCR can distinguish between modified (“mutant”) and unmodified (“wild-type”) genomic DNA. After three days of incubation in the presence of nanoparticles, genomic DNA from the human CD34⁺-derived cells were harvested to assess PNA-induced gene modification. In prior studies utilizing nucleofection for DNA and PNA delivery, it was shown that allele-specific PCR is a specific and reliable marker for targeted genomic modification, corresponding to altered mRNA splice products in the case of the targeted modification at the IVS2-1 splice site within beta-globin, as in the experiments here, and thus the same allele-specific PCR methods were used in this study. The same amount of genomic DNA was used for each PCR reaction. Allele-specific PCR showed that nanoparticle-delivered donor DNA was able to mediate site-specific modification, with highest levels of recombination in particles doubly loaded with PNA and DNA (PNA−DNA). qRT-PCR values for combined PNA−DNA particles and PNA−DNA nucleofection were 332 and 223 respectively, normalized to expression of β-globin wildtype allele.

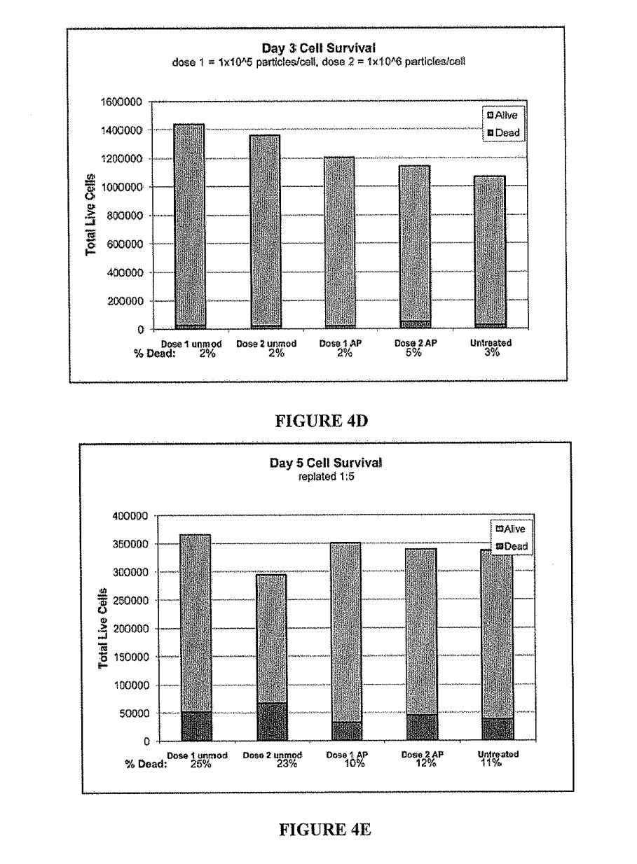

This oligonucleotide-mediated modification was dose-dependent, in that there was a higher level of genomic modification seen with cells treated with a high-dose of nanoparticles relative to cells treated with a medium-dose of nanoparticles, as demonstrated using quantitative real-time PCR on genomic DNA harvested after seven days of nanoparticle exposure (FIG. 6) Dosages are expressed as nmoles of nucleic acid/mL of media based on a particle loading of approximately 1 nmole nucleic acid/mg particles. For example, for “low” dose: 0.5 nmoles of DNA per mL media, or 0.5 nmoles DNA +0.5 nmoles PNA per mL media, based on attempted particle loading, which corresponds to 0.5 mg/mL DNA particles, 0.5 mg/mL DNA particles +0.5 mg/mL PNA particles, or 1 mg/mL PNA−DNA particles. “Medium”: 1 nmoles DNA per mL media, or 1 nmole DNA +1 nmole PNA per mL media. “High”: 2 nmoles DNA per mL media, or 2 nmole DNA +2 nmole PNA per mL media. Relative levels of modification are given in arbitrary units, with normalization to levels of β-globin wild-type primer amplification. Error bars where indicated give +/− standard deviation (n=3). Expression of the mutant is given in arbitrary units, with normalization to expression of the β-globin wildtype allele. PNA−DNA nucleofection and DNA nucleofection qRT-PCR values were 240,000±50,000 and 840,000±160,000, not shown in FIG. 6. PCR amplification with a gene-specific primer was used to verify similar genomic DNA loading.

To verify that the detection of gene modification using allele-specific PCR was not affected by the presence of residual donor DNA oligonucleotide in the PCR reaction, genomic DNA harvested from nanoparticle-treated CD34⁺ cells was electrophoresed to separate the high molecular weight genomic DNA from any residual oligonucleotide [Maurisse et al. (2006) Oligonucleotides 16: 375-386]. Allele-specific PCR of this gel-purified genomic DNA confirmed the presence of genomic modification, indicating that the observed PCR amplification did not arise as an artifact from possible contaminating oligonucleotides. In addition, a “spiking” experiment was preformed in which donor DNA oligonucleotide was added directly to genomic DNA harvested from untreated CD34⁺ cells, immediately prior to undergoing allele-specific PCR. No amplification using the mutant allele-specific primers was detected using semi-quantitative and quantitative PCR in these spiked samples, indicating that these donor oligonucleotides do not serve appreciably as PCR primers in the PCR reactions, and they do not participate in template-switching in this PCR assay.

Progenitor cells treated with nanoparticles were then differentiated into both erythroid and neutrophil populations with appropriate cytokines. Low-dose particle treated or nucleofected cells were grown in erythroid- or neutrophil-differentiating conditions, or in media with expansion (non-differentiating) cytokines (“expansion”), and routinely harvested for detection of presence of the β-globin mutant. FACS analyses of lineage-specific markers (CD34⁺ for progenitor population, glycophorin A for erythroid cells, and CD15 for neutrophils) of cells taken at various time points, up to 28 days post-treatment, were not significantly different among cells treated with oligonucleotide-containing nanoparticles, empty nanoparticles, and untreated cells. Nanoparticle-treated cells grown in either erythroid- or neutrophil-differentiating conditions also retained the gene modification as detected by allele-specific PCR up to 30 days following nanoparticle treatment.

To show the generalizability of this method, nanoparticle-mediated oligonucleotide delivery for the purpose of genomic modification was applied to another gene site, the human CCR5 gene, which encodes a chemokine receptor required for HIV-1 entry into human cells [Samson, M, et al. (1996) Nature 382: 722-725]. Nanoparticles were loaded with a single-stranded donor DNA, homologous to CCR5 except for a desired six nucleotide modification, along with a PNA that specifically targets CCR5. As above, human hematopoietic progenitor cells were incubated in medium containing nanoparticles loaded with either PNA plus DNA, or DNA alone, and harvested three days later to analyze for the site-specific modification.

Genomic DNA harvested from cells 3 days following nanoparticle treatment shows targeted modification at this alternate site. Plasmids containing the mutation or wild-type sequence of the human gene verify specificity of allele-specific primers. The same amount of genomic DNA was used for each PCR reaction. Blank (control) was CD34 cells treated with particles containing PBS only. Untreated (control) was CD34⁺ cells (cells in culture medium only). As for the β-globin target, modification levels were relatively higher in cells treated with nanoparticles containing both PNA and donor DNA, when compared with cells treated with DNA-only nanoparticles, again indicating that this PNA can augment modification at the genomic level in human CD34⁺ derived cells. Plasmid DNAs containing the 6-nucleotide mutation, or wild-type sequence, were used as a PCR control.

In summary, primary human CD34⁺ cells treated with PNA-containing nanoparticles exhibit low toxicity (Example 3, FIGS. 4A-E), and high levels of genomic recombination in (Example 4). Levels of genomic modification were higher in cells treated with nanoparticles containing both bis-PNA-194 and donor DNA, despite higher loading of nucleic acid in DNA-only particles, indicating that bis-PNA-194 was able to stimulate recombination of the donor DNA. The level of modification was dependent on nanoparticle dose, as shown by quantitative allele-specific PCR using genomic DNA harvested 7 days after treatment. Notably, cells that were co-treated with nanoparticles containing PNA and donor DNA separately (PNA+DNA) yielded a lower level of genomic modification, relative to cells treated with nanoparticles containing both PNA and donor DNA together (PNA−DNA). This higher level of modification correlates with the higher levels of oligonucleotide release in the combined particles, as compared with the separately PNA-loaded particles (Example 1, FIG. 1). In addition, the co-loaded nanoparticles may facilitate delivery of both PNA and DNA into individual cells; while treatment with separately loaded PNA and DNA particles relies on cells taking up two different nanoparticles independently in the same time-frame.

Of note, even DNA-only loaded nanoparticles caused detectable genomic modification in hematopoietic cells.

Long-term retention of the gene modification was demonstrated in nanoparticle-treated cells grown in either erythroid- or neutrophil-differentiating conditions. These results also indicate that nanoparticle and oligonucleotide treatment does not change the differentiation capacity of this cell population, and that the gene modification can persist throughout differentiation. In addition, the persistence of the modification up to 30 days indicates recombination in primitive cells. PNA-mediated modification at an additional gene site (CCR 5) by nanoparticle delivery, that the delivery method can be used at diverse target sites.

### Example 5

Gene Modification Frequency in CD34+ Cells Treated with PNAs and DNAs

### Materials and Methods

Estimation of Mutation Frequency by Plasmid Standard Curve

The human beta-globin gene was inserted into pcDNA4 (Invitrogen), and site-directed mutagenesis was used to insert the 6 basepair mutation at the IVS2 splice-site junction according the manufacturer's instructions (Invitrogen). Cloned and selected single colony plasmid DNA was sequenced to verify the presence of the human beta-globin gene and the expected mutation at IVS2. Serial dilutions of plasmid DNA were made in sterile nuclease-free water, and plasmid DNA concentrations were verified using spectroscopy at OD260. Known quantities of the mutant plasmid DNA were added to PCR reactions containing untreated (i.e. wild-type), purified CD34⁺ genomic DNA, at frequencies ranging from 0.014% to 14%.

Gene frequency was calculated as the number of copies of plasmid DNA divided by the estimated total number of cells constituting one PCR reaction. Quantitative PCR with mutant allele-specific primers was performed on the Stratagene MX2000 Pro Real-time PCR machine, and relative amplification values were calculated by subtracting the threshold cycle number from that of untreated CD34⁺ genomic DNA containing no plasmid (i.e. 0% frequency). These relative amplification values, normalized against values using wild-type specific primers, were plotted and fit to an exponential curve (Microsoft Excel). This fit curve was then used to calculate an estimated gene frequency for nucleofected- and nanoparticle-treated CD34⁺ genomic DNA, which were subjected to quantitative PCR at the same time for comparison.

Expansion of Limiting Dilution Cell Populations to Estimate Mutation Frequencies

As an independent assay to estimate genomic modification frequencies, CD34⁺ cells were treated with 2 mg/mL PNA−DNA particles or nucleofection with PNA and DNA as described above. Cells were then incubated for 3 days at 37° C. Following treatment, modification was confirmed with allele-specific PCR, and cell counts were performed with trypan blue to determine the number of live cells in each treatment group. For both particle-treated and nucleofected cells, cells were replated into 48 wells, with 20 cells/well each, in a 96 well plate in neutrophil expansion media. After 2 weeks, cells were split into two identical 96 well plates.

After 4 weeks, genomic DNA from one of the 96 well plates was then harvested using the Wizard SV 96 Genomic DNA purification system (Promega). Allele-specific PCR was performed as described above in 96-well format to determine the presence of genomic modification. Positive wells, as well as randomly selected negative wells, were then individually harvested from the second replica plate for verification using the Wizard Genomic DNA Purification kit and allele-specific PCR as described above.

A simple computation to determine a low-end estimate of modification frequency (# positive wells/48/20) yielded a modification frequency of 0.83% for particle-treated cells and 0.1% for nucleofected cells. A more robust calculation for the 95% confidence interval for frequency is described below.

Graphs and Statistical Analyses

Graphs were created using Microsoft Excel 2007. Data averaged for multiple samples is given as the mean +/− standard deviation (stdev). Determination of frequency using low dilution expansion was performed using Extreme Limiting Dilution Analysis (http://bioinf.wehi.edu.au/software/elda/index.html) [Hu, Y, and Smyth, G K (2009) J Immunol Methods 347: 70-78]. Briefly, a limiting dilution assay assumes the Poisson single-hit model: the number of positive hits (in this case, modification) in a culture varies with a Poisson distribution, and a single positive cells in a culture is sufficient to produce a positive response (in this case, at least 1 of the 20 cells plated in a well of the 96-well plate). The estimates and 95% confidence intervals are given in the Results section.

### Results

Quantification of the frequency of targeted gene modification in CD34⁺-derived cells following introduction of PNA and donor DNA by nanoparticle treatment or by nucleofection was also determined. In one approach, a standard curve of mutation frequency was generated using quantitative allele-specific PCR of known quantities of plasmid DNA containing the mutant form of the human beta-globin gene, mixed with genomic DNA from mock-transfected human CD34⁺ cells. The plasmid-based beta-globin gene was altered by site-directed mutagenesis to contain the same 6 base-pair mutation as that introduced by the donor DNA oligonucleotide.

Quantitative AS-PCR using primers specific for the introduced mutation was performed on genomic DNA from particle-treated or nucleofected CD34⁺ cells, and relative values (normalized to wild-type AS-PCR, n=3) were compared to a standard curve generated by quantitative AS-PCR with known amounts of mutant plasmid copies. Increasing amounts of pcDNA4 with the mutant human beta-globin gene, containing the same modification as that introduced by the donor DNA oligonucleotide, were added to wild-type genomic DNA from untreated CD34⁺ cells, and subjected to quantitative AS-PCR using primers specific for the targeted modification. The resulting normalized values were plotted against the calculated mutant allele frequency, generating a standard curve that was used to estimate modification frequencies of nanoparticle- and nucleofected-CD34⁺ samples (depicted by square and triangle symbols, respectively).

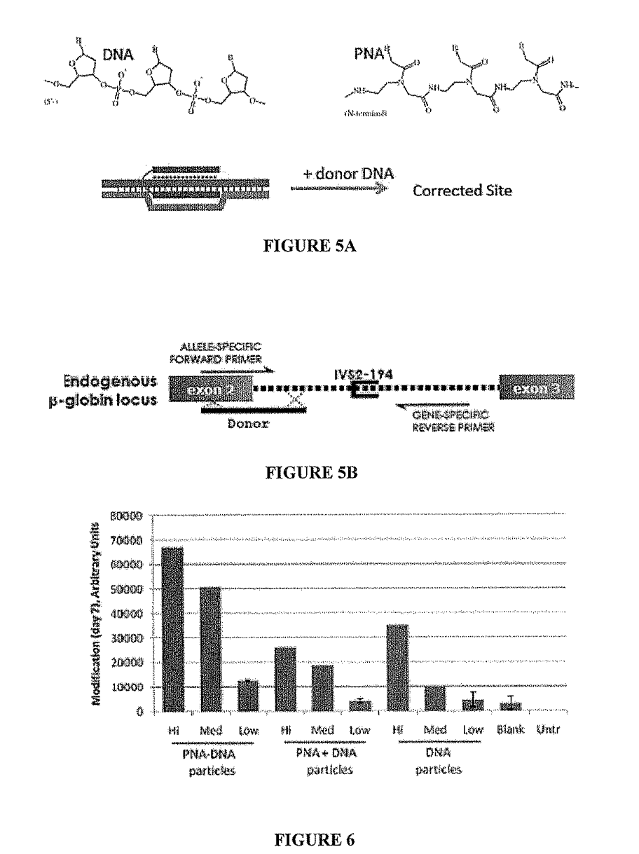

After generating a standard curve with mutant copies ranging from 20 to 20,000, representing genomic mutation frequencies of 0.01% to 14% in 600 ng of genomic DNA, the relative PCR amplification values using genomic DNA harvested from nanoparticle-treated or nucleofected CD34⁺ cells were compared to estimate a mutant gene frequency following oligonucleotide treatment. Using this method, the estimated frequency of gene modification was 0.2% for cells treated with nanoparticles, and 0.05% for cells treated by Amaxa nucleofection (FIG. 7). The circle symbol denotes a PCR sample in which purified wild-type genomic DNA was spiked with donor DNA oligonucleotide immediately prior to the PCR reaction. This control PCR reaction was to assure that the presence of single stranded DNA donor oligonucleotides would not serve as artifact for the mutant AS-PCR reaction.

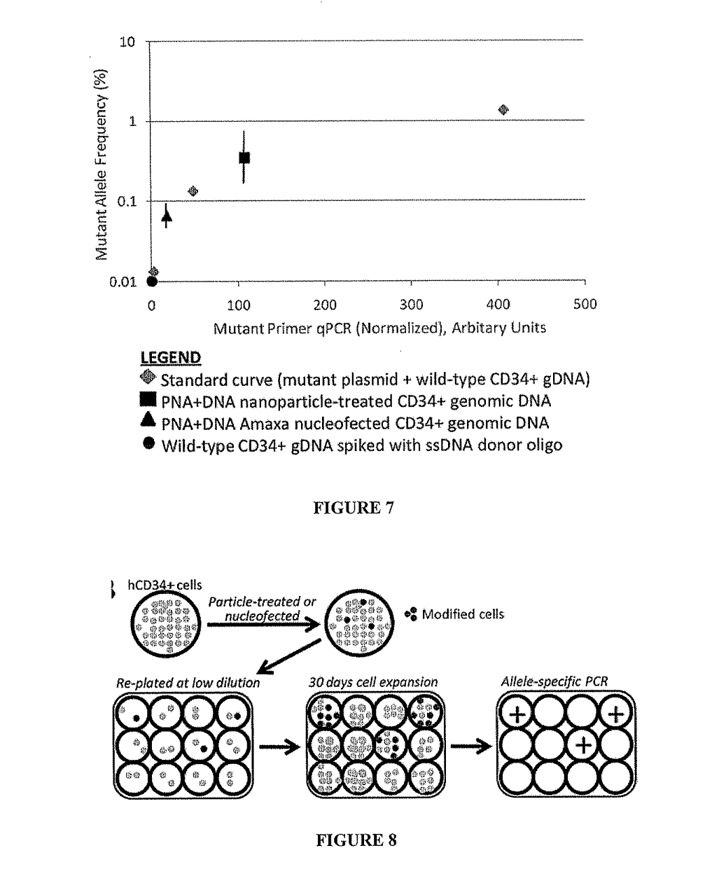

To confirm this finding, an independent method was used to quantify the frequency of genomic modification based on analysis of clonal populations following limiting dilution. Because of the difficulty of growing single human CD34+ cells to large enough populations to perform PCR, limiting dilution was performed. Human primary CD34⁺ cells were treated with 2 mg/mL PNA−DNA particles or nucleofection as above, and plated at low dilution (20 cells/well, 48 wells each). A schematic of the experimental design is shown in FIG. 8. After one month of expansion in neutrophil-promoting conditions, the individual cell populations were harvested for genomic DNA, and presence of the modification in each well was determined using allele-specific PCR. A well was counted as positive if the mutation was detectable by allele-specific PCR. It was found that 8 of 48 wells were positive for the particle-treated, whereas only one out of the 48 was positive for the nucleofected cells. Using a single-hit Poisson model (Extreme Limiting Dilution Analysis http://bioinf.wehi.edu.au/software/elda/index.html), the estimated recombination frequencies were 0.91% (95% confidence intervals 0.46%-1.82%) for the particle-treated cells and 0.11% (95% confidence interval 0.01-0.74%) for the nucleofected cells, statistically overlapping with the range seen with the plasmid standard (Table 2).

TABLE 2
Comparison of estimated modification frequencies

| hCD34+ cell treatment | Std Curve qPCR | Limiting dilution |
|---|---|---|
| Nucleofection | 0.05% | 0.10% |
| (95% confidence intervals) | (0.03-0.11%) | (0.01-0.74%) |
| PNA-DNA nanoparticles | 0.2% | 0.91% |
| (95% confidence intervals) | (0.08-0.4%) | (0.46-1.82%) |

In summary, quantification indicates that particle-treatment resulted in greater recombination frequencies than obtained by nucleofection, with targeted modification of the β-globin gene in the range of 0.46-1.82% in a single treatment as determined in a limiting dilution clonal assay. If one million cells are treated with nucleofection, combining the survival data (Example 3) and percentage of observed gene modification at day 3 (Example 4), 16,000 total modified and viable cells are available. In contrast, 1,008,000 modified cells are available after particle treatment using this same calculation, a 63-fold increase.

We claim:

1. A method for increasing efficiency and decreasing cytotoxicity of delivery of mutagenic or recombinagenic nucleic acid molecules comprising providing the nucleic acid molecules encapsulated into polymeric particles.

2. The method of claim 1 wherein the nucleic acid molecules are encapsulated to a weight percentage of between 0.01 and 5% of the polymer.

3. The method of claim 1 wherein the nucleic acid molecules are selected from the group consisting of triplex forming molecules, donor molecules, and combinations thereof.

4. The method of claim 3 wherein the nucleic acid molecules are donor molecules.

5. The method of claim 3 wherein the nucleic acid molecules are donor molecules in combination with triplex forming molecules, and wherein the triplex forming molecules are triplex forming peptide nucleic acids.

6. The method of claim 3 wherein the nucleic acid molecules are donor molecules in combination with triplex forming molecules, and wherein the triplex forming molecules are triplex forming oligonucleotides.

7. The method of claim 3 wherein the nucleic acid molecules are triplex forming molecules, and wherein the triplex forming molecules are triplex forming oligonucleotides.

8. The method of claim 7 wherein the nucleic acid molecules are triplex forming molecules, and wherein the triplex forming molecules are psoralen-linked triplex forming oligonucleotides.

9. The method of claim 1 wherein the polymeric particles are sized to promote encocytosis of the particles by the cell which is to be modified by the triplex forming nucleic acid molecules.

10. The method of claim 1 wherein the particles have targeting molecules on their surfaces to direct to the particles to specific target cells.

11. The method of claim 1 wherein the particles are between about 10 nm and 1000 nm, preferably about 50 nm and 500 nm, most preferably between about 100 nm and 200 nm.

12. The method of claim 1 wherein the particles are targeted to phagocytic cells.

13. The polymeric particles having triplex forming nucleic acid molecules encapsulated therein of claim 1.


SEQUENCE LISTING (3 sequences)

SEQ ID NO: 1 - protein, 6 aa, Artificial Sequence
  Synthetic Peptide Nucleic Acid
LLLLLL

SEQ ID NO: 2 - DNA, 13 bp, Artificial Sequence
  Synthetic Peptide Nucleic Acid
tatgacatgaact

SEQ ID NO: 3 - DNA, 50 bp, Artificial Sequence
  Synthetic DNA oligonucleotide
aaacatcaagggtcccataggtctattctgaagttctcaggatccacgtg